(19) United States
(12) Patent Application Publication
(10) Pub. No.: US 2025/0000927 A1
(43) Pub. Date: Jan. 2, 2025

(54) ADAPTOGEN FORMULATIONS AND METHODS OF USE

(71) Applicant: MDM Enterprise Solutions Inc., Menlo Park, CA (US)

(72) Inventors: Eleanor DUFF, Portola Valley, CA (US); Kevin SPELMAN, Ashland, OR (US); Scott EAGLE, Menlo Park, CA (US); John ROZELLE, Los Angeles, CA (US)

(73) Assignee: MDM Enterprise Solutions Inc., Menlo Park, CA (US)

(21) Appl. No.: 18/760,639

(22) Filed: Jul. 1, 2024

Related U.S. Application Data
(63) Continuation-in-part of application No. PCT/US2023/064654, filed on Mar. 17, 2023.
(60) Provisional application No. 63/323,365, filed on Mar. 24, 2022.

(51) Int. Cl. A61K 36/258; A61K 31/00; A61K 31/198; A61K 31/221; A61K 31/7068; A61K 36/185; A61K 36/28; A61K 36/41; A61K 36/48; A61K 36/66; A61K 36/77; A61K 36/79; A61K 36/81

(52) U.S. Cl. CPC A61K 36/258; A61K 31/198; A61K 31/221; A61K 31/658; A61K 31/7068; A61K 36/185; A61K 36/28; A61K 36/41; A61K 36/48; A61K 36/66; A61K 36/77; A61K 36/79; A61K 36/81

(57) ABSTRACT

Described herein are nutritional compositions comprising one or more adaptogens, medicinal botanicals, nutraceuticals, and combinations thereof. The disclosed compositions improve focus, energy, and stress conditions. Also provided are methods for increasing mental focus, increasing mental energy and physiological energy, and reducing mental stress.

### FIELD OF THE DISCLOSURE

The present disclosure provides nutritional compositions comprising one or more adaptogens, medicinal botanicals, nutraceuticals, and combinations thereof, for focus, energy, stamina, motivation, sociability, and recovery from stress, thereby facilitating the Flow State. The adaptogens in each precision formulation work through synergistically increasing mental focus, mental and physiological energy, and reducing stress while boosting the drive to accomplish tasks, and reducing physical and mental tension and pain while boosting emotional balance and calm. The brainwave paradigm (alpha, beta, delta, gamma, and theta) and neurotransmitter profiles are also affected, which reflects the transition to the Flow State. The adaptogen formulations of the disclosure can also be combined with cannabis to provide a controlled and directed cannabis experience by synergizing the positive benefits and mitigating the negative effects of cannabis.

### BACKGROUND OF THE INVENTION

The compositions of the present invention are constructed using multiple approaches involving plant-based medicine, and the endocannabinoid system.

Plant-based medicines, also called phytomedicines, are well-tolerated, with less severe as well as a smaller range of side effects compared with many synthetic drugs. In addition, while synthetic drugs are based upon a single biologically active ingredient, many phytomedicines exert beneficial effects through the actions of several biologically active compounds acting at single and/or multiple target sites. Additionally, these systems have generated a model of constructing complex phytochemical matrices that include synergistic activation of biochemical pathways that converge on a common therapeutic/pharmacological outcome. Thus, plant compounds can have overlapping pharmacological pleiotropy in that a single plant compound acts on multiple targets and, therefore, they provide multiple effects on different biochemical pathways to converge on a common beneficial biochemical and therapeutic outcome.

Adaptogens are herbs and plants that, when consumed, can help the body adapt to various stresses. Adaptogens also exhibit overlapping pharmacological pleiotropy. While each type of adaptogen has unique properties and benefits, adaptogenic substances generally have the capacity to normalize body functions and strengthen systems compromised by stress. Adaptogens have protective effects on health against a wide variety of environmental assaults, as well as both physical and psychological stressors.

Network pharmacology is applied to construct each formulation by targeting multiple but select pharmacological targets. By carefully building each formula, lower doses are needed for each target, any potential for adverse events is reduced, and desired pharmacological outcomes are amplified. The model generated through a novel combination of these approaches is Network Pharmacology (NP).

The endocannabinoid system (ECS) is also an important element in promoting resilience in response to stressors. The ECS is an integrated system of receptors, ligands, and enzymes that regulate fundamental processes in the central and peripheral nervous systems of the body. The recognition of the ECS is relatively new, and the number of targets included in this physiological network continues to expand beyond the cannabinoid receptor 1 and 2 (CB1 and CB2). See, e.g., Iannotti et al., Prog Lipid Res., 62:107-128 (2016). Cannabinoids are compounds found in Cannabis plants that target various aspects of the ECS. Notable cannabinoids include cannabidiol (CBD), cannabinol (CBN), and tetrahydrocannabinol (THC)—the compound that produces the euphoric “high” from cannabis consumption.

Cannabis has also been identified as a rejuvenating herb that, when consumed, can promote health and prevent disease. Indeed, cannabis consumption can have positive effects on, e.g., sleep, anxiety, mood, creativity, focus, and energy levels. However, differences in cannabinoid profiles between Cannabis plant varietals, coupled with differences in the physiology, metabolism, and mental state between individual consumers on any given day, can provide inconsistent results from person to person when consuming cannabinoid compositions. For example, depending on the Cannabis cultivar and the individual consumer, consumption of a particular cannabis product may cause one person to feel energetic while the same product causes a different person to feel sleepy. Or a product may cause one person to feel relaxed and comfortable while the same product causes a different person to feel anxious and paranoid.

In addition, chronic overstimulation of the ECS can have negative effects. Signaling by the ECS is known to regulate the hypothalamic-pituitary-adrenocortical (HPA) axis in the context of a stress response. Ziegler C G, et al., Horm Metab Res. 2010 February; 42 (2): 88-92. Chronic overstimulation of the ECS, such as through chronic consumption of cannabis products, can have negative effects on the HPA axis and homeostasis within the body. For example, studies have shown that chronic cannabis consumption can case blunted adrenocorticotropic hormone (ACTH) and cortisol reactivity in response to acute stress, resulting in a dysregulated HPA axis. HPA axis dysfunction can manifest in numerous symptoms, including fatigue, disturbed sleep, anxiety, immunosuppression, adrenal suppression, hypocortisolism, adverse effect on brain function, including learning and memory, and changes in body weight.

Thus, there is a need for more effective botanical formulations that provide consistent beneficial effects on the mind and body, while avoiding and even reducing negative effects on the HPA axis. The compositions of the present disclosure meet this need. Moreover, the compositions of the present disclosure surprisingly act as a cannabinoid adjuvant by enhancing the effectiveness of cannabinoids on the ECS. These surprising and unexpected results include reduction of gastrointestinal stress response, reduction in feelings of social isolation and fear of peer judgment, reduction in avoidance and coping patterns that lead to social detachment, and reduction in brain fog and morning fatigue after cannabis consumption. The compositions of the present disclosure provide an improved outcome with cannabinoid usage through several modes of activity.

Unexpected benefits from drug and/or plant combinations can occur due to interconnecting signaling networks within and between cells. Such benefits have been observed when treating complex network-driven diseases such as cancer, multiple sclerosis, parasites, or infectious diseases (e.g., antifungals and antibiotic combinations for targeting infectious diseases). Although a Network Pharmacology analysis is useful, such an analysis cannot reliably predict the outcomes of drug and/or or plant combinations when different metabolic pathways or drug targets are concurrently activated. The compositions of the present disclosure achieved several unexpected benefits. For example, (1) a stress recovery formulation of the present disclosure unexpectedly reduced the GI stress response, (2) a sociability formulation of the present disclosure unexpectedly reduced feelings of social isolation and fear of peer judgement, (3) a stamina and motivation formulation of the present disclosure unexpectedly reduced avoidance and coping patterns leading to social detachment, and (4) a focus formulation of the present disclosure unexpectedly reduced the Cannabis hangover, particularly by reducing brain fog and morning fatigue.

The compositions of the present disclosure are also useful in achieving a psychological state known as the “Flow State.” The Flow State is a holistic sensation felt by an individual when that individual acts with complete involvement in an activity. The Flow State is characterized by a sense of intrinsic reward experienced during immersive engagement in an activity, and is associated with motivation, peak performance, and peak experience and enjoyment. The Flow State is achieved when an individual becomes so completely involved in an activity that they lose track of time and their surroundings, with the exception of the activity itself.

Physically, the Flow State can be identified by specific changes in (1) neural oscillations (brain waves) and (2) neurotransmitters. In the Flow State, (1) neural oscillations comprise increased alpha and theta frequencies and (2) neural transmitters primarily comprising norepinephrine and dopamine, and secondarily comprising serotonin, anandamide (increasing the tone of the endocannabinoid system), and adrenocorticotropic hormone (ACTH)—are released at increased levels.

The Flow State impacts many different types of activities, such as sports and athletic performance, scientific learning, work productivity, engineering (e.g., software engineering for gaming), chess playing, rock climbing, and dancing. In addition to the sense of intrinsic reward experienced during an activity, objective measures of individuals in the Flow State demonstrate gains in athletic performance, musical performance, dance, and eSports. Additionally, individuals achieving the Flow State exhibit increases in motivation and skill development. For example, athletes in the Flow State often push themselves to new and higher performance limits.

A variety of factors can influence the case/frequency with which individuals enter the Flow State. For example, self-esteem, neurotocism, extraversion, employment status, differences in dopamine receptors, and genetics can impact the case/frequency with which individuals enter the Flow State. Regarding genetics, the case/frequency of entering the Flow State has an estimated 41% heritability.

Nonetheless, elite athletes report that experiencing the Flow State is controllable. Indeed, there is a greater likelihood of experiencing the Flow State when individuals (1) focus their attention, (2) improve their confidence, and (3) quiet their self-criticality. Accordingly, the compositions of the present disclosure are useful for inducing neurological conditions for facilitating the Flow State. Accordingly, through facilitating the Flow State, the compositions of the present disclosure can enhance performance in many areas.

### BRIEF SUMMARY OF THE INVENTION

The present disclosure provides nutritional compositions comprising an effective amount of one or more adaptogens, medicinal botanicals, nutraceuticals, or a combination thereof. In some aspects, the compositions comprise Withania somnifera, Schisandra chinensis, Rhodiola rosea, Panax quinquefolius, Panax ginseng, or any combination thereof.

In some aspects, the composition of the present disclosure comprise about 200 mg to about 700 mg of Withania somnifera, about 250 mg to about 400 mg of Schisandra chinensis, about 100 mg to about 400 mg of Rhodiola rosea, about 40 mg to about 300 mg of Panax quinquefolius, about 40 mg to about 300 mg of Panax ginseng, or any combination thereof.

In some aspects, the compositions of the present disclosure comprise one or more medicinal botanicals are Paullinia cupana, Sceletium tortuosum, Ilex paraguariensis, Griffonia simplicifolia, Corydalis yanhusuo, Silybum marianum, Piper methysticum, Salvia rosmarinus, Melissa officinalis, Paullinia cupana, Passiflora incarnata, or any combination thereof.

In some aspects, the compositions of the present disclosure comprise one or more of the following about 90 mg to about 270 mg of Paullinia cupana, about 5 mg to about 200 mg of Sceletium Tortuosum, about 100 mg to about 250 mg of Ilex paraguariensis, about 20 mg to about 200 mg of Griffonia simplicifolia, about 100 mg to about 300 mg of Corydalis yanhusuo, about 30 mg to about 140 mg of Silybum marianum, about 25 mg to about 100 mg of Piper methysticum, about 200 mg to about 400 mg of Salvia rosmarinus, about 50 mg to about 150 mg of Melissa officinalis, about 90 mg to about 270 mg of Paullinia cupana, about 50 mg to about 300 mg of Passiflora incarnata, or any combination thereof.

In some aspects, the compositions of the present disclosure comprise one or more nutraceuticals are acetyl-L-carnitine, citicoline, L-theanine, or any combination thereof.

In some aspects, the compositions of the present disclosure comprise one or more of the following about 100 mg to about 400 mg of acetyl-L-carnitine, about 100 mg to about 400 mg of citicoline, about 100 mg to about 300 mg of L-theanine or any combination thereof.

In some aspects, the compositions of the present disclosure comprise about 40 mg to about 300 mg of Panax quinquefolius, about 100 mg to about 400 mg of acetyl-L-carnitine, about 100 mg to about 400 mg of citicoline, and about 100 mg to about 300 mg of L-theanine.

In some aspects, the compositions of the present disclosure comprise about 40 mg to about 300 mg of Panax ginseng, about 100 mg to about 400 mg of acetyl-L-carnitine, about 100 mg to about 400 mg of citicoline, and about 100 mg to about 300 mg of L-theanine.

In some aspects, the compositions of the present disclosure comprise about 200 mg to about 700 mg of Withania somnifera, about 100 mg to about 300 mg of Corydalis yanhusuo, about 30 mg to about 140 mg of Silybum marianum and about 20 mg to about 200 mg of Griffonia simplicifolia.

In some aspects, the compositions of the present disclosure comprise about 100 mg to about 400 mg of Rhodiola rosea, about 90 mg to about 270 mg of Paullinia cupana, and about 5 mg to about 200 mg of Sceletium Tortuosum.

In some aspects, the compositions of the present disclosure comprise about 200 mg to about 700 mg of Withania somnifera, about 250 mg to about 400 mg of Schisandra chinensis, about 100 mg to about 250 mg of Ilex paraguariensis and about 20 mg to about 200 mg of Griffonia simplicifolia.

In some aspects, the compositions of the present disclosure further comprise hemp oil, one or more cannabinoids, or any combination thereof.

The present disclosure also provides methods of alleviating hypothalamic-pituitary-adrenal (HPA) axis dysfunction in a subject, comprising administering to the subject one or more compositions of the disclosure. In some aspects, HPA axis dysfunction occurs as a result of a subject's cannabis consumption. In some aspects, the HPA axis dysfunction in the subject manifests as fatigue, disturbed sleep, anxiety, immunosuppression, adrenal suppression, hypocortisolism, adrenocorticotropic hormone (ACTH) suppression, changes in body weight, or any combination thereof.

The present disclosure also provides methods of enhancing the effects of one or more cannabinoids in a subject, comprising administering to the subject one or more compositions of the disclosure, wherein the composition acts as a cannabinoid adjuvant.

In some aspects, the one or more cannabinoids are tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, trihydroxy-delta-9-tetrahydrocannabinol, or any combination thereof.

In some aspects, the one or more cannabinoids is CBD, CBN, THC, or any combination thereof.

The present disclosure also provides methods for increasing mental focus in a subject, comprising administering to the subject a composition comprising about 40 mg to about 300 mg of Panax quinquefolius, about 100 mg to about 400 mg of acetyl-L-carnitine, about 100 mg to about 400 mg of citicoline, and about 100 mg to about 300 mg of L-theanine. In some aspects, the method further comprise administering to the subject a composition comprising one or more cannabinoids.

The present disclosure also provides methods for increasing mental energy and physiological energy in a subject, comprising administering to the subject a composition comprising about 100 mg to about 400 mg of Rhodiola rosea, about 90 mg to about 270 mg of Paullinia cupana, and about 5 mg to about 200 mg of Sceletium Tortuosum. In some aspects, the method further comprise administering to the subject a composition comprising one or more cannabinoids.

The present disclosure also provides methods for increasing stamina and motivation in a subject, comprising administering to the subject a composition comprising about 100 mg to about 400 mg of Rhodiola rosea, about 90 mg to about 270 mg of Paullinia cupana, and about 5 mg to about 200 mg of Sceletium Tortuosum. In some aspects, the method further comprise administering to the subject a composition comprising one or more cannabinoids.

The present disclosure also provides methods for increasing sociability in a subject, comprising administering to the subject a composition comprising about 200 mg to about 700 mg of Withania somnifera, about 250 mg to about 400 mg of Schisandra chinensis, about 100 mg to about 250 mg of Ilex paraguariensis and about 20 mg to about 200 mg of Griffonia simplicifolia. In some aspects, the method further comprise administering to the subject a composition comprising one or more cannabinoids.

The present disclosure also provides methods for reducing environmental, physical, and mental stress in a subject, comprising administering to the subject a composition about 200 mg to about 700 mg of Withania somnifera, about 100 mg to about 300 mg of Corydalis yanhusuo, about 30 mg to about 140 mg of Silybum marianum and about 20 mg to about 200 mg of Griffonia simplicifolia. In some aspects, the method further comprise administering to the subject a composition comprising one or more cannabinoids.

### Embodiments

Embodiment 1. A nutritional composition comprising an effective amount of one or more adaptogens.

Embodiment 2. The composition of embodiment 1, wherein the one or more adaptogens are selected from Withania somnifera, Schisandra chinensis, Rhodiola rosea, Corydalis yanhusuo, Griffonia simplicifolia, Ilex paraguariensis, Piper methysticum, Salvia rosmarinus, Melissa officinalis, Paullinia cupana, Panax quinquefolius, Passiflora incarnate, Sceletium tortuosum, or any combination thereof

Embodiment 3. The composition of embodiments 1 or 2, comprising one or more of the following: about 200 mg to about 700 mg of Withania somnifera, about 200 mg to about 400 mg of Salvia rosmarinus, about 100 mg to about 300 mg of Corydalis yanhusuo, about 5 mg to about 100 mg of Griffonia simplicifolia, about 0.1 mg to about 1 mg of Melissa officinalis essential oil, about 100 mg to about 300 mg of Panax quinquefolius, about 90 mg to about 270 mg of Paullinia cupana, about 25 mg to about 100 mg of Piper methysticum, about 50 mg to about 150 mg of Melissa officinalis, about 50 mg to about 300 mg of Passiflora incarnate, about 100 mg to about 400 mg of Rhodiola rosea, about 5 mg to about 200 mg of Sceletium tortuosum, about 250 mg to about 400 mg of Schisandra chinensis, or about 100 mg to about 250 mg of Ilex paraguariensis.

Embodiment 4. The composition of embodiment 2, further comprising citicoline, L-theanine, or any combination thereof.

Embodiment 5. The composition of embodiment 4, comprising about 100 mg to about 300 mg of Panax quinquefolius, about 150 mg to about 400 mg of citicoline, and about 100 mg to about 200 mg of L-theanine.

Embodiment 6. The composition of embodiment 3, comprising about 200 mg to about 400 mg of Salvia rosmarinus, about 100 mg to about 300 mg of Panax quinquefolius, and about 5 mg to about 200 mg of Sceletium tortuosum.

Embodiment 7. The composition of embodiment 3, comprising about 5 mg to about 200 mg of Sceletium tortuosum, about 90 mg to about 270 mg of Paullinia cupana, and about 100 mg to about 400 mg of Rhodiola rosea.

Embodiment 8. The composition of embodiment 3, comprising about 200 mg to about 700 mg of Withania somnifera, about 250 mg to about 400 mg of Schisandra chinensis, and about 100 mg to about 250 mg of Ilex paraguariensis.

Embodiment 9. The composition of 3, comprising about 200 mg to about 700 mg of Withania somnifera, about 0.1 mg to about 1 mg of Melissa officinalis essential oil, about 50 mg to about 150 mg of Melissa officinalis, and about 50 mg to about 300 mg of Passiflora incarnate.

Embodiment 10. The composition of 3, comprising about 200 mg to about 700 mg of Withania somnifera, and about 25 mg to about 100 mg of Piper methysticum.

Embodiment 11. The composition of embodiment 3, comprising about 5 mg to about 100 mg of Griffonia simplicifolia, about 200 mg to about 700 mg of Withania somnifera, and about 100 mg to about 300 mg of Corydalis yanhusuo.

Embodiment 12. The composition of any one of embodiments 1 to 11, further comprising hemp oil.

Embodiment 13. The composition of any one of embodiments 1 to 12, further comprising at least one flavorant.

Embodiment 14. A method of alleviating hypothalamic-pituitary-adrenal (HPA) axis dysfunction in a subject, comprising administering to the subject the composition of any one of embodiments 1 to 13.

Embodiment 15. The method of embodiment 14, wherein the HPA axis dysfunction in the subject is a result of the subject's cannabis consumption.

Embodiment 16. The method of embodiments 14 or 15, wherein the HPA axis dysfunction in the subject manifests as fatigue, disturbed sleep, anxiety, immunosuppression, adrenal suppression, hypocortisolism, adrenocorticotropic hormone (ACTH) suppression, changes in body weight, or any combination thereof.

Embodiment 17. A method of increasing mental focus in a subject, comprising administering to a subject a composition comprising about 200 mg to about 400 mg of Salvia rosmarinus, about 100 mg to about 300 mg of Panax quinquefolius, about 30 mg to about 200 mg of Sceletium tortuosum, about 150 mg to about 400 mg of citicoline, about 100 mg to about 200 mg of L-theanine, or any combination thereof.

Embodiment 18. The method of embodiment 17, wherein the composition comprises about 200 mg to about 400 mg of Salvia rosmarinus, about 100 mg to about 300 mg of Panax quinquefolius, and about 30 mg to about 200 mg of Sceletium tortuosum.

Embodiment 19. The method of embodiment 17, wherein the composition comprises a about 100 mg to about 300 mg of Panax quinquefolius, about 150 mg to about 400 mg of citicoline and about 100 mg to about 200 mg of L-theanine.

Embodiment 20. The method of any one of embodiments 17 to 19, wherein the composition further comprises hemp oil.

Embodiment 21. The method of any one the embodiments 17 to 20, further comprising administering to the subject a composition comprising one or more cannabinoids.

Embodiment 22. A method of increasing mental energy and physiological energy in a subject, comprising administering to a subject a composition comprising about 90 mg to about 270 mg of Paullinia cupana, about 5 mg to about 200 mg of Sceletium tortuosum, about 100 mg to about 400 mg of Rhodiola rosea, about 200 mg to about 700 mg of Withania somnifera, about 250 mg to about 400 mg of Schisandra chinensis, and about 100 mg to about 250 mg of Ilex paraguariensis.

Embodiment 23. The method of embodiment 22, wherein the composition comprises about 90 mg to about 270 mg of Paullinia cupana, about 5 mg to about 200 mg of Sceletium tortuosum, and about 100 mg to about 400 mg of Rhodiola rosea.

Embodiment 24. The method of embodiment 22, wherein the composition comprises about 200 mg to about 700 mg of Withania somnifera, about 250 mg to about 400 mg of Schisandra chinensis, and about 100 mg to about 250 mg of Ilex paraguariensis.

Embodiment 25. The method of any one of embodiments 22 to 24, wherein the composition further comprises hemp oil.

Embodiment 26. The method of any one of the embodiments 22 to 25, further comprising administering to the subject a composition comprising one or more cannabinoids.

Embodiment 27. A method of reducing mental stress in a subject, comprising administering to the subject a composition comprising about 5 mg to about 100 mg of Griffonia simplicifolia, about 200 mg to about 700 mg of Withania somnifera, about 0.1 mg to about 1 mg of Melissa officinalis essential oil, about 50 mg to about 150 mg of Melissa officinalis, about 50 mg to about 300 mg of Passiflora incarnate, about 25 mg to about 100 mg of Piper methysticum, and about 100 mg to about 300 mg of Corydalis yanhusuo.

Embodiment 28. The method of embodiment 27, wherein the composition comprises about 200 mg to about 700 mg of Withania somnifera, about 0.1 mg to about 1 mg of Melissa officinalis essential oil, about 50 mg to about 150 mg of Melissa officinalis, and about 50 mg to about 300 mg of Passiflora incarnate.

Embodiment 29. The method of embodiment 28, wherein the composition comprises about 200 mg to about 700 mg of Withania somnifera, and about 25 mg to about 100 mg of Piper methysticum.

Embodiment 30. The method of embodiment 28, wherein the composition comprises about 5 mg to about 100 mg of Griffonia simplicifolia, about 200 mg to about 700 mg of Withania somnifera, and about 100 mg to about 300 mg of Corydalis yanhusuo.

Embodiment 31. The method of any one of embodiments 27 to 30, wherein the composition further comprises hemp oil.

Embodiment 32. The method of any one of the embodiments 27 to 31, further comprising administering to the subject a composition comprising one or more cannabinoids.

Embodiment 33. The method of any one of embodiments 31, 26, or 32, wherein the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

Embodiment 34. The method of embodiment 33, wherein the cannabinoid is CBD, CBN, THC, or any combination thereof.

### BRIEF DESCRIPTION OF THE FIGURES

FIG. 1 shows the network pharmacology for an exemplary composition designed for increasing mental focus.

FIG. 2 shows the network pharmacology for an exemplary composition designed to increase stamina, motivation, and mental and physiological energy.

FIG. 3 shows the network pharmacology for an exemplary composition designed for increasing sociability.

FIG. 4 shows the network pharmacology for an exemplary composition designed to increase recovery from stress.

FIG. 5 is a bar graph depicting consumer responses related to mental and cognitive benefits after consuming exemplary compositions of the present disclosure.

FIG. 6 is a bar graph depicting consumer responses related to physical benefits after consuming exemplary compositions of the present disclosure.

FIG. 7 is a pie chart depicting consumer responses after consuming exemplary compositions of the disclosure.

FIG. 8 is a bar graph depicting consumer responses related to stress recovery after consuming exemplary compositions of the present disclosure.

FIG. 9 is a bar graph depicting consumer positive experiences after consuming exemplary compositions designed for increasing mental focus.

FIG. 10 is a bar graph depicting consumer positive experiences after consuming exemplary compositions designed for increasing energy.

FIG. 11 shows the involvement of neural oscillations (brain waves) and neurotransmitters for an exemplary composition for increasing mental focus.

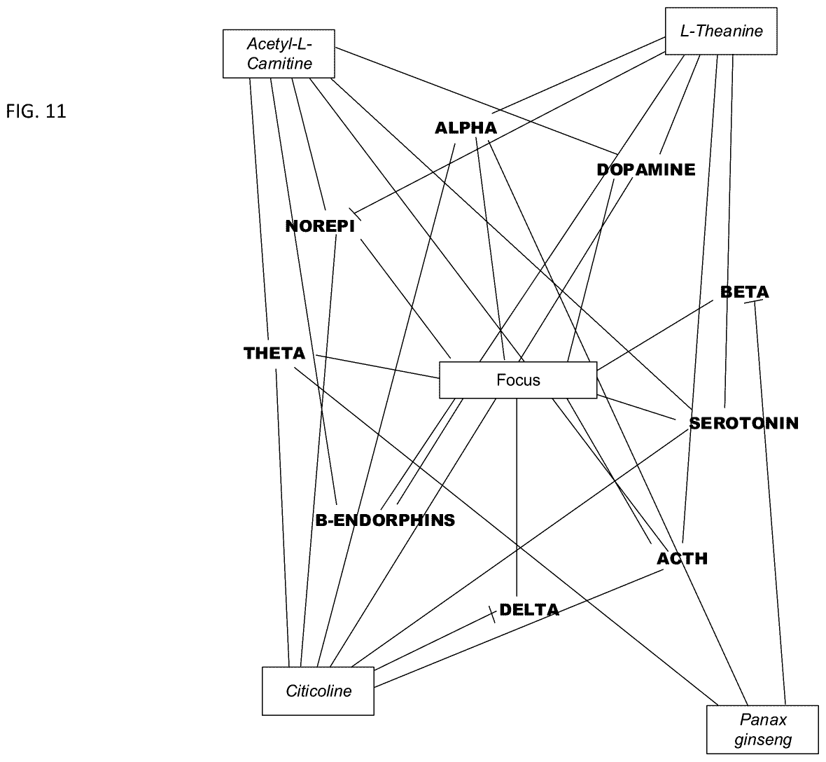

FIG. 12 shows the involvement of neural oscillations (brain waves) and neurotransmitters for an exemplary composition for increasing stamina, motivation, and mental and physiological energy.

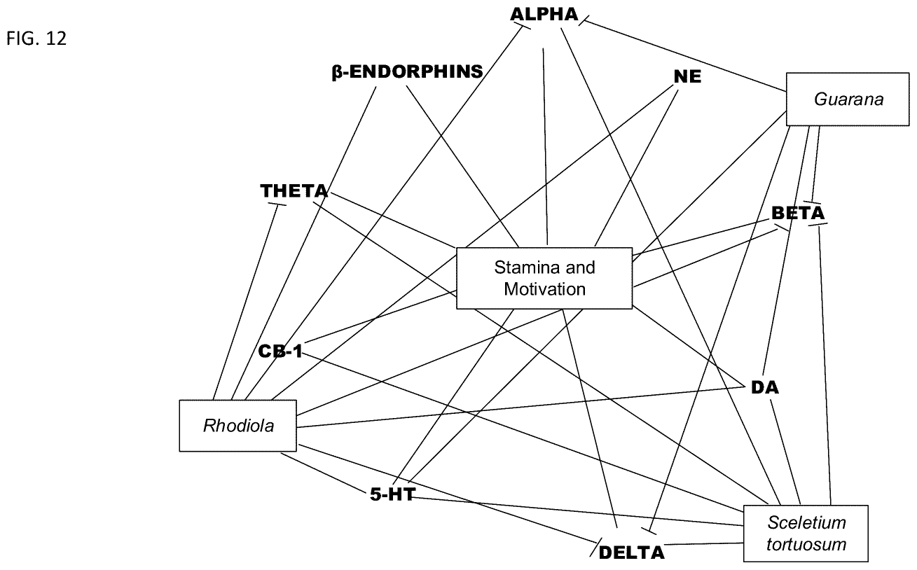

FIG. 13 shows the involvement of neural oscillations (brain waves) and neurotransmitters for an exemplary composition for increasing sociability.

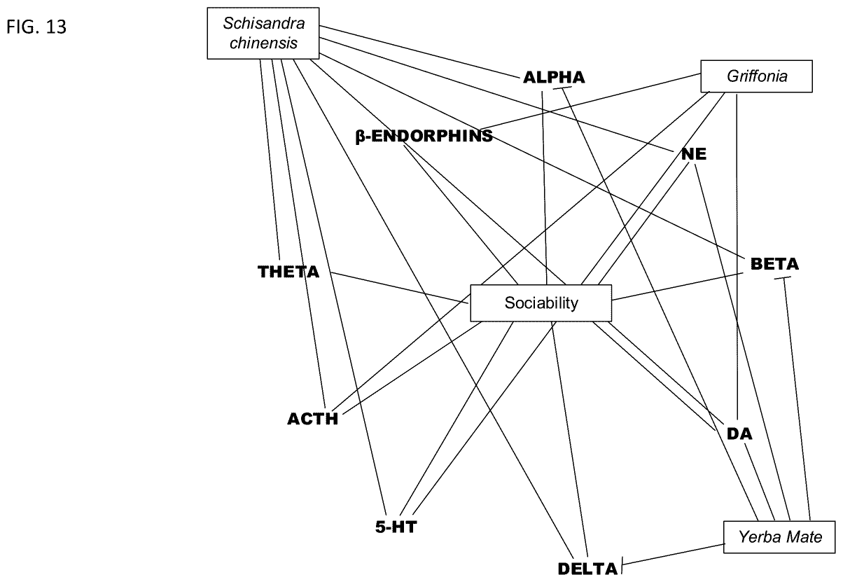

FIG. 14 shows the involvement of neural oscillations (brain waves) and neurotransmitters for an exemplary composition for increasing recovery from stress.

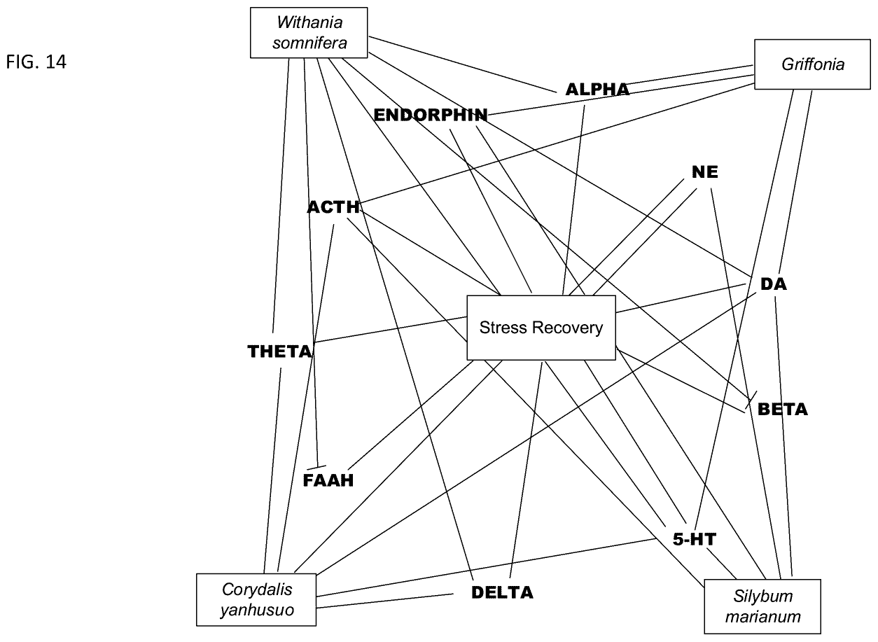

### DETAILED DESCRIPTION OF THE INVENTION

### I. General Definitions

Unless defined otherwise, all technical and scientific terms used herein have the same meaning as commonly understood by one of ordinary skill in the art to which this invention belongs. In case of conflict, the present application including the definitions will control. Additional definitions are set forth throughout the application. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

As used herein, the singular form “a”, “an” and “the” include plural references unless the context clearly dictates otherwise. For example, the term “an ingredient” or “at least one ingredient” can include a plurality of ingredients, including mixtures thereof.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specified otherwise, all of the designations “A %-B %,” “A-B %,” “A % to B %,” “A to B %,” “A %-B,” “A % to B” are given their ordinary and customary meaning. In some aspects, these designations are synonyms.

It is understood that wherever aspects are described herein with the language “comprising,” otherwise analogous elements described in terms of “consisting of” and/or “consisting essentially of” are also provided.

As used herein, the terms “comprises”, “comprising”, “includes”, “including”, “having,” and their conjugates mean “including but not limited to.”

As used herein, the term “consisting of” means “including and limited to.”

As used herein, the term “consisting essentially of” means the specified material of a composition, or the specified steps of a method, and those additional materials or steps that do not materially affect the basic characteristics of the material or method.

When the term “about” is used in conjunction with a numerical value or range, it modifies that value or range by extending the boundaries above and below the numerical values set forth. The term “about” is used herein to modify a numerical value above and below the stated value by a variance of 10 percent, up or down (higher or lower), i.e., ±10%, unless a different variance is indicated (e.g., ±30%, ±20%, ±5%, ±1%, etc.).

As used herein, the term “process” and “method” refer to manners, means, techniques, and procedures for accomplishing a given task including, but not limited to, those manners, means, techniques, and procedures either known to or readily developed from known methods, means, techniques and procedures by practitioners of the chemical, pharmacological, biological, biochemical and medical arts.

The term “composition” as described herein refers to a combination of ingredients (e.g., plant-based botanicals, such adaptogen). The terms “composition” and “formulation” are used interchangeably herein

The terms “administer,” “administering,” “administration,” and the like, as used herein, refer to the administration of a composition (e.g., a composition as described herein) to a subject or system. Administration to an animal subject (e.g., to a human) can be by any appropriate route, such as one described herein.

The terms “subject” and “individual”, as included herein, are used interchangeably. None of the terms are to be interpreted as requiring the supervision of a medical professional (e.g., a doctor, nurse, physician's assistant, orderly, hospice worker).

The term “symptom” or “clinical symptom” refers to subjective evidence of a disease, such as a feeling of nausea, as perceived by the subject.

As used herein, the term “effective amount” or “therapeutically effective amount” of a composition, extract, extract mixture, component of the extract, and/or active agent or ingredient refers to an amount effective at dosages and for periods of time sufficient to achieve a desired result. For example, the “effective amount” or “therapeutically effective amount” refers to that amount of a composition, extract, extract, extract mixture, botanical extract mixture, component of the extract, and/or active agent or ingredient of this disclosure which, when administered to a subject (e.g., a human), is sufficient to achieve the desired beneficial effect treatment, such as increased focus, reduced stress, or increased energy, compared to a subject not administered the composition.

Throughout this application, various aspects of this disclosure can be presented in a range format. It should be understood that the description in range format is merely for convenience and brevity and should not be construed as an inflexible limitation on the scope of the disclosure.

Accordingly, the definition of a range should be considered to have specifically disclosed all the possible subranges and individual numerical values within that range. For example, description of a range, such as from 1 to 6, should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5 and 6. This applies regardless of the breadth of the range.

“Percent” or “%” as used herein refers to a weight percentage (w/w) unless otherwise specified.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is appreciated that certain features of the disclosure, which are, for clarity, described in the context of particular aspects, can also be provided in combination in a single aspect. Conversely, various features of the disclosure, which are, for brevity, described in the context of a single aspect, can also be provided separately or in any suitable sub-combination or as suitable in any other described aspect of the disclosure. Certain features described in the context of various aspects are not considered essential features unless the aspect is inoperative without those elements.

The compositions of the present disclosure are prepared based on the principles of pharmacological convergence. The term “pharmacological convergence” refers to synergistic activation of biological pathways intersecting with molecular targets to result in a specific pharmacological outcome. The compositions of the present disclosure apply a model of constructing complex phytochemical matrices that include synergistic interactions. By triggering select biochemical pathways and molecular targets, convergence of disparate biological pathways on a pharmacological outcome is achieved.

II. Composition for Focus, Energy, Stamina, Motivation, Sociability, and Recovery from Stress.

The present disclosure provides nutritional compositions comprising an effective amount of one of more adaptogens complimented by herbal and chemical supporting active ingredients and plant-based adjuvants. The present disclosure provides nutritional compositions comprising an effective amount of one or more adaptogens, medicinal botanicals, and nutraceuticals for increasing mental focus, improving mental energy and physiological energy, improving stamina, improving motivation, improving sociability and recovery from stress.

Adaptogens are herbs and plants that can help the body “adapt” to various biological stressors. While each adaptogen has unique properties and benefits, adaptogens generally (i) are innocuous agents, nonspecifically increasing resistance against physically, chemically, biologically, and psychologically noxious factors (“stressors”) normalizing effect independent of the nature of pathologic state; (ii) support homeostasis in the body; and (iii) are reported to have a protective effect on health against a wide variety of environmental assaults and emotional conditions. See e.g., II. B. [Eleutherococcus]. Nauka; 1968:168; Wagner H, Nörr H, Winterhoff H. Phytomedicine. June 1994; 1 (1): 63-76.

Adaptogens affect the hypothalamic-pituitary-adrenal (HPA axis) in response to stimulation by external stress. Chronic stress exposure, due to varying stressors, often results in HPA axis dysregulation in a wide variety of individuals. HPA axis dysfunction manifests in a variety of ways as symptoms are often non-specific and may include, but are not limited to, weakness, fatigue, malaise, nausea, abdominal pain, poor weight gain, headache, anergia, disturbed sleep, anxiety, immunosuppression, adrenal suppression, hypocortisolism, adrenocorticotropic hormone (ACTH) suppression, and changes in body weight.

Adaptogens are known for their ability to maintain or recover homeostasis and allostasis in the body, as well as promote anabolic recovery. Adaptogens can produce positive responses to stress through regulation of key mediators of the stress response such as, stress-activated c-Jun N-terminal protein kinase (JNK1), β-endorphin, nitric oxide and HPA-axis, including cortisol and glucocorticoid receptors. See, e.g., Panossian A, Wikman G. Pharmaceuticals (Basel). 2010; 3 (1): 188-224. The HPA axis maintains physiological homeostasis and as such regulates many bodily processes thus, adaptogens exhibit polyvalent beneficial effects against chronic inflammation, atherosclerosis, neurodegenerative cognitive impairment, metabolic disorders, cancer, and other aging-related diseases. See, e.g., Panossian, A. (2017). Ann. N.Y. Acad. Sci., 1401:49-64.

Adaptogens exhibit multi-target action and the shared use of several receptors, including corticosteroid, mineralocorticoid, progestin, estrogen, serotonin (5-HT), N-methyl-d-aspartate, and nicotinic acetylcholine, receptor tyrosine kinases, and many G protein-coupled receptors. See, e.g., Panossian, A. (2017). Ann. N.Y. Acad. Sci., 1401:49-64. The multi-target effects on the neuroendocrine-immune system include but is not limited to:
i. Triggering of intracellular and extracellular adaptive signaling pathways that promote cell survival and organismal resilience in stress;
ii. Regulation of metabolism and homeostasis via effects on expression of stress hormones (corticotrophin and gonadotropin-releasing hormones, urocortin, cortisol, neuropeptide Y, heat shock proteins Hsp70) and their receptors.

In some aspects, the adaptogens of the present disclosure comprise phenolic compounds or tetracyclic triterpenoids.

Phenolic compounds include, but are not limited to, phenylpropanoids and phenylethane derivatives, such as salidroside (rhodioloside), rosavin, syringin, triandrin, tyrosol, and lignans, such as eleutherosid E and schisandrin B. These compounds are structurally similar to the catecholamines, which mediate the sympathoadrenal system involved in activation of the stress system in the early stages of a stress response.

Tetracyclic triterpenoids include, but are not limited to, cucurbitacin R diglucoside, ginsenosides, and phytosterol-glycosides (e.g., SG, eleutheroside A, sitoindosides, and daucosterol). These compounds structurally resemble the corticosteroids that act as stress hormones involved in the protective inactivation of the stress system.

In some aspects, the compositions of the present disclosure are prepared by applying network pharmacology. Network pharmacology can provide treatments for complex diseases, chronic conditions, and syndromes, inclusive of their pathophysiologic evolution, through a synergistic effect of the compositions comprising adaptogens according to the disclosure. Network Pharmacology when applied to compounding formulas can also attenuate adverse events induced by one or more herbs in a formula or an additional agent taken alongside a compounded formula.

The phrase “synergistic effect” refers to an interaction between two or more compounds that causes the total effect of the compounds to be greater than the sum of the individual effects of each compound. For example, two or more adaptogens in the compositions of the present disclosure may have a synergistic effect on increasing mental focus, increasing mental energy and physiological energy, or reducing stress. In some aspects, the compositions according to the present disclosure increase sociability, increase motivation, and/or recovery from the effects of environmental, physical, and/or psychological stress. In some aspects, the compositions of the disclosure further comprise one or more hemp-derived oils. In some aspects, the compositions of the present disclosure combined with a composition comprising one or more cannabinoids may have a synergistic effect on increasing mental focus, increasing mental energy and physiological energy, or reducing stress. In some aspects, the compositions of the disclosure further comprise one or more hemp-derived oils.

In some aspects, the compositions of the present disclosure are prepared by combining Eastern and Western philosophies, botanicals, and supplements to target multiple targets that help regulate mental focus, energy levels, and responses to stress. As used herein, “botanicals” refers to a substance obtained from a plant and used as an additive.

Example botanicals include, but are not limited to, ashwagandha, holy basil, ginseng, rhodiola, chamomile, lemon balm, gotu kola, bacopa, schisandra, gingko, spearmint, Nicotiana mutabilis, turmeric, Lion's Mane, Salvia officinalis, Salvia lavandulifolia, and any combinations thereof. Exemplary forms of a medicinal botanical include, but are not limited to, a dried botanical, an extract, a powder, a concentrate, and any combinations thereof. As used herein, “nutraceuticals” refers to a substance containing health-giving additives and having medicinal benefit.

In some aspects, the compositions of the present disclosure are cannabinoid adjuvants. As used herein, the term “cannabinoid adjuvant” refers to a substance or combination of substances that are used to increase the efficacy or potency of cannabinoids on the endocannabinoid system.

In some aspects, the compositions of the present disclosure are prepared on the principle of pharmacological convergence. By applying a model of constructing complex phytochemical matrices that include synergist and triggering select biochemical pathways, convergence of disparate biochemical pathways on a pharmacological outcome is achieved.

In some aspects, the compositions of the present disclosure are prepared by applying an overlapping pharmacological pleiotropy, which is also a strategy used by Eastern medicine. As used herein, the term “pharmacological pleiotropy” refers to a plant compound having multiple targets and, thus, numerous effects on physiology.

The partial perturbations of medicinal plants on a pharmacological network mimic physiological scenarios where hundreds of different enzyme systems and receptor types and subtypes act in concert. See e.g., Ágoston V. et al., Phys Rev E., 71 (5): 1-8 (2005). Some studies of cellular networks demonstrate that pharmacological agents that provide multiple signals can impact the complex equilibrium of whole cellular networks more favorably than drugs that act on a single target. See, e.g., Csermely P. et al., Trends Pharmacol Sci., 26 (4): 178-182 (2005); Keith, C. T. & Zimmermann, G. R., Current Drug Discovery. 19-23 (2004); Morphy R. et al., Drug Discov Today, 9 (15): 641 (2004); Briskin D P. Plant Physiol., 124 (2): 507-514 (2000); Ágoston V. et al., Phys Rev E., 71 (5): 1-8 (2005).

Additionally, multi-target agents may partially affect their targets, which corresponds well with the presumed low-affinity interactions of medicinal plants. See e.g., Ágoston V. et al., Phys Rev E., 71 (5): 1-8 (2005). Broader specificity, lower affinity, multi-component compounds have been reported to be, in some cases, more efficient than high affinity, high specificity compounds. See, e.g., Csermely P., Trends Biochem Sci., 29 (7): 331-334 (2004); Lipton S A., Nature. 428 (6982): 473 (2004); Vickers A, et al., BMJ. 319 (7216): 1050-1053 (1999).

Other advantages of low-affinity, multi-target pharmacological strategies include improved safety profiles (lower occurrence and reduced range of side effects) compared with high affinity, single-target drugs. See, e.g., Ideker T. et al., Trends Biotechnol., 21 (6): 255 (2003); Jia W. et al., Phytother Res., 18 (8): 681-686 (2004); Shu H H, et al., J Ethnopharmacol. 103 (3): 398-405 (2006); Hikino H, et al., J Pharmacobiodyn. 3 (10): 514-525 (1980); Suzuki Y. et al., Planta Medica., 60 (5): 391-394 (1994).

Described herein are the compositions comprising an effective amount of one or more adaptogens, medicinal botanicals and/or nutraceuticals. In some aspects, the compositions of the disclosure comprise an effective amount of one or more adaptogens, medicinal botanicals and/or nutraceuticals wherein at least one adaptogenic compound provides neuroprotection, mood support and regulates key biological systems such as metabolism and homeostasis via impact on the expression of stress hormones (corticotrophin and gonadotropin-releasing hormones, urocortin, cortisol, neuropeptide Y, heat shock proteins Hsp70) and their receptors.

In some aspects, the one or more adaptogens are selected from Withania somnifera, Schisandra chinensis, Rhodiola rosea, Panax quinquefolius, Aralia elata, Asphaltum bitumen, Cordyceps sinesis, Cynomorium coccineum, Ganoderma lingzhi, Morinda citrifolia, Ocimum tenuiflorum, Rhaponticum carthamoides, or any combination thereof. In some aspects, the one or more adaptogens are selected from Withania somnifera, Schisandra chinensis, Rhodiola rosea, Piper methysticum, Salvia rosmarinus, Melissa officinalis, Panax quinquefolius, Passiflora incarnata, or any combination thereof. In some aspects, the one or more adaptogens are selected from Withania somnifera, Schisandra chinensis, Rhodiola rosea, Panax quinquefolius, Panax ginseng, or any combination thereof.

In some aspects, the one or more medicinal botanicals are selected from Paullinia cupana, Sceletium tortuosum, Ilex paraguariensis, Griffonia simplicifolia, Corydalis yanhusuo, Silybum marianum, ashwagandha, holy basil, ginseng, rhodiola, lemon balm, gotu kola, bacopa, schisandra, gingko, spearmint, Nicotiana mutabilis, tumeric, Lion's Mane, choline, Piracetam, Salvia officinalis, Salvia lavandulifolia or any combination thereof. In some aspects, the one or more medicinal botanicals are selected from Paullinia cupana, Sceletium tortuosum, Ilex paraguariensis, Griffonia simplicifolia, Piper methysticum, Salvia rosmarinus, Melissa officinalis, Passiflora incarnata, Corydalis yanhusuo, Silybum marianum, or any combination thereof.

In some aspects, the one or more medicinal botanicals are selected from Paullinia cupana, Sceletium tortuosum, Ilex paraguariensis, Griffonia simplicifolia, Corydalis yanhusuo, Silybum marianum, or any combination thereof.

In some aspects, the one or more nutraceuticals are selected from acetyl-L-carnitine, citicoline, choline, piracetam, L-theanine, creatine, or any combination thereof. In some aspects, the one or more nutraceuticals are selected from acetyl-L-carnitine, citicoline, L-theanine, or any combination thereof.

The compositions of the disclosure are designed to employ the protective and synergistic effects of adaptogens to not only increase mental focus, increase mental energy and physiological energy, increase sociability, increase motivation, and recover from the effects of environmental, physical, and/or psychological stress, but also to attenuate the effects of stressors while directing and controlling the strain-specific effects of Cannabis sativa L. As used herein, the term “strain-specific” refers to the effects of individual sub-species of Cannabis that give the buds distinct aromas and effects. For example, some strain-specific effects may involve relieving pain, while another may involve inducing sleep, while another may involve increasing energy.

In some aspects, the compositions of the present disclosure work synergistically with the consumption of cannabis to provide a more controlled cannabis experience in a subject, guided by the formulation of the adaptogen composition. For example, an adaptogen composition of the disclosure formulated for increasing mental energy and physiological energy may work synergistically with a cannabis product to provide the subject with more controlled feelings of energy and vigor. Or, an adaptogen composition of the disclosure formulated for recovering from stress may work synergistically with a cannabis product to provide the subject with more controlled feelings of relaxation and well-being. In some aspects, the disclosed methods provide for a more controlled cannabis experience in a subject while concurrently alleviating HPA axis dysfunction in the subject.

### Compositions for Increasing Mental Focus

The present disclosure provides compositions for increasing mental focus. Mental focus includes cognitive functions such as concentration and attention. Attention is the ability to actively process specific information in the environment while tuning out other details. The attentional control center resides in both the prefrontal and parietal lobes in the brain. Moreover, neuromodulators such as acetylcholine, dopamine, serotonin, and norepinephrine play an important role in attention and focus. See, e.g., Palacios-Filardo J, Mellor J R. Curr Opin Neurobiol. 2019 February; 54:37-43.

In some aspects, the compositions of the disclosure increase mental focus. In some aspects, the compositions of the disclosure increase attention. In some aspects, the compositions of the disclosure increase concentration. In some aspects, the compositions of the disclosure increase attention and concentration.

In some aspects, the compositions of the present disclosure alleviate adverse effects of cannabis consumption in a subject. Cannabis has been known to increase confusion in some subjects, and it may result in reversible deficits in cognition, particularly short-term memory. See, e.g., Walsh Z, et al. Clin Psychol Rev. February 2017; 51:15-29. Thus, the compositions of the present disclosure can offset these issues using adaptogen formulations based on their network pharmacology and predicted effects on these brain regions through the regulation of the multiple proteins involved in cognition.

In some aspects, compositions of the present disclosure for increasing mental focus comprise an effective amount of Salvia rosmarinus, Panax quinquefolius, Sceletium tortuosum, citicoline, L-theanine, or any combination thereof. In certain aspects, the compositions of the present disclosure comprise about 200 mg to about 400 mg of Salvia rosmarinus, about 100 mg to about 300 mg of Panax quinquefolius, about 5 mg to about 200 mg of Sceletium tortuosum, about 150 mg to about 400 mg of citicoline, about 100 mg to about 200 mg of L-theanine, or any combination thereof.

In some aspects, the compositions of the present disclosure comprise about 200 mg to about 400 mg of Salvia rosmarinus, about 100 mg to about 300 mg of Panax quinquefolius, and about 5 mg to about 200 mg of Sceletium tortuosum.

In certain aspects, the compositions of the present disclosure comprise Salvia rosmarinus in the amount of about 200 mg, about 205 mg, about 210 mg, about 215 mg, about 220 mg, about 225 mg, 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, 330 mg, about 335 mg, about 340 mg, about 345 mg, about 350 mg, about 355 mg, about 360 mg, about 365 mg, about 370 mg, about 375 mg, about 380 mg, about 385 mg, about 290 mg, about 395 mg, about 400 mg. In certain aspects, the compositions of the present disclosure comprise about 200 mg of Salvia rosmarinus. In certain aspects, the compositions of the present disclosure comprise 200 mg of Salvia rosmarinus. In certain aspects, the compositions of the present disclosure comprise about 250 mg of Salvia rosmarinus. In certain aspects, the compositions of the present disclosure comprise 250 mg of Salvia rosmarinus. In certain aspects, the compositions of the present disclosure comprise about 300 mg of Salvia rosmarinus. In certain aspects, the compositions of the present disclosure comprise 300 mg of Salvia rosmarinus. In certain aspects, the compositions of the present disclosure comprise about 400 mg of Salvia rosmarinus. In certain aspects, the compositions of the present disclosure comprise 400 mg of Salvia rosmarinus.

Salvia is a genus of about 1000 species of shrubs, herbaceous perennials, and annuals in the sage family, Lamiaceae, distributed throughout the Old World and the Americas. See, e.g., Walker, J. B. et al. (2004), Am. J. Bot., 91:1115-1125; Clebsch, Betsy; Barner, Carol D. (2003). The New Book of Salvias. Timber Press. ISBN 978-0-88192-560-9.

Salvia species of the present disclosure include, but are not limited to, Salvia rosmarinus, Salvia officinalis, Salvia miltiorrhiza, Salvia apiana, Salvia azurea, and Salvia buchananii.

Salvia rosmarinus, formerly known as Rosmarinus officinalis, is commonly known as rosemary and contains several phytochemicals, including rosmarinic acid, camphor, caffeic acid, ursolic acid, betulinic acid, carnosic acid, and carnosol. See e.g. Vallverdú-Queralt, et al. (2014). Food Chemistry. 154:299-307. S. rosmarinus inhibits the phosphorylation of mitogen-activated protein kinases (MAPKs), thereby blocking nuclear factor kappa beta (NF-kB) activation, which in turn leads to decreased expression of inducible nitric oxide synthase (iNOS) and cyclooxygenase-2 (COX-2), thus preventing inflammation. See, e.g., Yu M H, et al. Food Chem. 2013 Jan. 15; 136 (2): 1047-54. In addition, S. rosmarinus exhibits neurotrophic effects and improved cholinergic functions in correlation with MAPK, ERK1/2 signaling pathway. Moreover, S. rosmarinus treated cells had significant upregulation of tyrosine hydroxylase (TH) and pyruvate carboxylase (PC), two major genes involved in dopaminergic, serotonergic, and GABAergic pathway regulations. See, e.g., Sasaki K et al. Behav Brain Res. 2013 Feb. 1; 238:86-94. S. rosmarinus has also demonstrated acetylcholinesterase inhibition, thus having the potential to increase acetylcholine in brain. See, e.g., Kamli M R, et al., Plants (Basel). 2022 Feb. 14; 11 (4): 514.

In some aspects, the compositions of the present disclosure comprise a species of Panax plant. Panax is a genus of about 17 species of perennial plants in the ivy family, Araliaceae, distributed throughout Asia and the Americas. See e.g., Boopathi V. et al., J Ginseng Res., 44 (1): 33-43 (2020).

Panax species of the present disclosure include, but are not limited to, Panax quinquefolius (American ginseng), Panax notoginseng, Panax bipinnatifidus, Panax japonicas, Panax ginseng (Asian ginseng, Chinese ginseng, Korean ginseng), Panax sokpayensis, Panax vietnamensis, Panax wangianus, Panax zingiberensis, Panax pseudoginseng, Panax stipuleanatus, and Panax trifolius.

Panax quinquefolius, better known as American ginseng, contains dammarane-type ginsenosides, or saponins. Panax quinquefolius contains a high level of Rb1 ginsenosides upregulates serotonin and dopamine levels in the brain. Moreover, this ginsenoside upregulates the expression of neurotrophic factors, thereby regulating the HPA axis's function. As well as, showing positive benefits in cognition and executive function in clinical trials.

In certain aspects, the compositions of the present disclosure comprise Panax quinquefolius or Panax ginseng in the amount of about 40 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg. In some aspects, the compositions of the present disclosure comprise about 70 to about 90 mg of Panax quinquefolius or Panax ginseng. In certain aspects, the compositions of the present disclosure comprise about 85 mg of Panax quinquefolius or Panax ginseng. In certain aspects, the compositions of the present disclosure comprise 85 mg of Panax quinquefolius or Panax ginseng. In certain aspects, the compositions of the present disclosure comprise about 100 mg of Panax quinquefolius or Panax ginseng. In certain aspects, the compositions of the present disclosure comprise 100 mg of Panax quinquefolius or Panax ginseng. In certain aspects, the compositions of the present disclosure comprise about 150 mg of Panax quinquefolius or Panax ginseng. In certain aspects, the compositions of the present disclosure comprise 150 mg of Panax quinquefolius or Panax ginseng. In certain aspects, the compositions of the present disclosure comprise about 200 mg of Panax quinquefolius or Panax ginseng. In certain aspects, the compositions of the present disclosure comprise 200 mg of Panax quinquefolius or Panax ginseng. In certain aspects, the compositions of the present disclosure comprise about 250 mg of Panax quinquefolius or Panax ginseng. In certain aspects, the compositions of the present disclosure comprise 250 mg of Panax quinquefolius or Panax ginseng. In certain aspects, the compositions of the present disclosure comprise about 300 mg of Panax quinquefolius or Panax ginseng. In certain aspects, the compositions of the present disclosure comprise 300 mg of Panax quinquefolius or Panax ginseng.

In certain aspects, the compositions of the present disclosure comprise Sceletium tortuosum in the amount of about 5 mg, about 10 mg, about 15 mg, about 20 mg, about 25 mg, about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, 160 mg, about 165 mg, about 170 mg, about 175 mg, about 180 mg, about 185 mg, about 190 mg, about 195 mg, about 200 mg. In certain aspects, the compositions of the present disclosure comprise about 50 mg of Sceletium tortuosum. In certain aspects, the compositions of the present disclosure comprise 50 mg of Sceletium tortuosum. In certain aspects, the compositions of the present disclosure comprise about 75 mg of Sceletium tortuosum. In certain aspects, the compositions of the present disclosure comprise 75 mg of Sceletium tortuosum. In certain aspects, the compositions of the present disclosure comprise about 100 mg of Sceletium tortuosum. In certain aspects, the compositions of the present disclosure comprise 100 mg of Sceletium tortuosum. In certain aspects, the compositions of the present disclosure comprise about 200 mg of Sceletium tortuosum. In certain aspects, the compositions of the present disclosure comprise 200 mg of Sceletium tortuosum.

Sceletium species of the present disclosure include, but are not limited to, Sceletium tortuosum, Sceletium emarcidum, Sceletium exalatum, Sceletium expansum, Sceletium rigidum, Sceletium strictum, Sceletium crassicaule, and Sceletium varians. See, e.g., Harvey A L, et al., J Ethnopharmacol. 2011 Oct. 11; 137 (3): 1124-9.

Sceletium tortuosum, recently known as Mesembryanthemum tortuosum, is a succulent plant commonly found in South Africa, it is widely known as ‘Kanna’. See, e.g., B.-E. Van Wyk, B. Gorelik, South African Journal of Botany, 2017, Volume 110, Pages 18-38. Sceletium tortuosum contains 25 alkaloids that display a broad spectrum of biological activity, e.g., anti-oxidant, immunomodulatory, neuroprotection, etc. See e.g., Olatunji T L, et al., J Ethnopharmacol. 2021 Nov. 15; 280:114476. Sceletium tortuosum increases serotonin levels by inhibiting its reuptake or upregulation of the monoamine transporter. See, e.g., Coetzee D D, et al., J Ethnopharmacol. 2016 Jan. 11; 177:111-6.

In some aspects, compositions of the present disclosure comprise an effective amount of Salvia rosmarinus, Panax quinquefolius, Sceletium tortuosum, or any combination thereof. In particular aspects, the compositions comprise an effective amount of Salvia rosmarinus, Panax quinquefolius, and Sceletium tortuosum. In some aspects, the compositions of the present disclosure comprise about 200 mg to about 400 mg of Salvia rosmarinus, about 100 mg to about 300 mg of Panax quinquefolius, and about 30 mg to about 200 mg of Sceletium tortuosum. In particular aspects, the composition comprises 300 mg of Salvia rosmarinus, 200 mg of Panax quinquefolius, and 100 mg of Sceletium tortuosum.

In some aspects, compositions of the present disclosure comprise an effective amount of Panax quinquefolius, citicoline, L-theanine, or any combination thereof. In particular aspects, compositions of the present disclosure comprise an effective amount of Panax quinquefolius, citicoline, and L-theanine. In certain aspects, the compositions of the present disclosure comprise about 100 mg to about 300 mg of Panax quinquefolius, about 150 mg to about 400 mg of citicoline, and about 100 mg to about 200 mg of L-theanine.

In certain aspects, the compositions of the present disclosure comprise Panax quinquefolius in the amount of about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg. In certain aspects, the compositions of the present disclosure comprise about 100 mg of Panax quinquefolius. In certain aspects, the compositions of the present disclosure comprise 100 mg of Panax quinquefolius. In certain aspects, the compositions of the present disclosure comprise about 150 mg of Panax quinquefolius. In certain aspects, the compositions of the present disclosure comprise 150 mg of Panax quinquefolius. In certain aspects, the compositions of the present disclosure comprise about 200 mg of Panax quinquefolius. In certain aspects, the compositions of the present disclosure comprise 200 mg of Panax quinquefolius. In certain aspects, the compositions of the present disclosure comprise about 250 mg of Panax quinquefolius. In certain aspects, the compositions of the present disclosure comprise 250 mg of Panax quinquefolius. In certain aspects, the compositions of the present disclosure comprise about 300 mg of Panax quinquefolius. In certain aspects, the compositions of the present disclosure comprise 300 mg of Panax quinquefolius. In certain aspects, the compositions of the present disclosure comprise about 100 mg of Panax ginseng. In certain aspects, the compositions of the present disclosure comprise 100 mg of Panax ginseng. In certain aspects, the compositions of the present disclosure comprise about 150 mg of Panax ginseng. In certain aspects, the compositions of the present disclosure comprise 150 mg of Panax ginseng. In certain aspects, the compositions of the present disclosure comprise about 200 mg of Panax ginseng. In certain aspects, the compositions of the present disclosure comprise 200 mg of Panax ginseng. In certain aspects, the compositions of the present disclosure comprise about 250 mg of Panax ginseng. In certain aspects, the compositions of the present disclosure comprise 250 mg of Panax ginseng. In certain aspects, the compositions of the present disclosure comprise about 300 mg of Panax ginseng. In certain aspects, the compositions of the present disclosure comprise 300 mg of Panax ginseng.

In certain aspects, the compositions of the present disclosure comprise Citicoline in the amount of about 150 mg, 160 mg, about 170 mg, about 180 mg, about 190 mg, 200 mg, about 210 mg, about 220 mg, 230 mg, about 240 mg, about 250 mg, about 260 mg, about 270 mg, about 280 mg, about 290 mg, about 300 mg, about 310 mg, about 320 mg, 330 mg, about 340 mg, about 350 mg, about 360 mg, about 370 mg, about 380 mg, about 390 mg, or about 400 mg. In certain aspects, the compositions of the present disclosure comprise about 150 mg of citicoline. In certain aspects, the compositions of the present disclosure comprise 150 mg of citicoline. In certain aspects, the compositions of the present disclosure comprise about 200 mg of citicoline. In certain aspects, the compositions of the present disclosure comprise 200 mg of citicoline. In certain aspects, the compositions of the present disclosure comprise about 250 mg of citicoline. In certain aspects, the compositions of the present disclosure comprise 250 mg of citicoline. In certain aspects, the compositions of the present disclosure comprise about 300 mg of citicoline. In certain aspects, the compositions of the present disclosure comprise 300 mg of citicoline. In certain aspects, the compositions of the present disclosure comprise about 400 mg of citicoline. In certain aspects, the compositions of the present disclosure comprise 400 mg of citicoline.

Citicoline, also known as cytidine diphosphate-choline (CDP-Choline) or cytidine 5′-diphosphocholine, is an intermediate in the generation of phosphatidylcholine from choline, a common biochemical process in cell membranes. Citicoline increases the availability of neurotransmitters, including acetylcholine, norepinephrine, and dopamine. See e.g. Secades J J, Lorenzo J L. Methods Find Exp Clin Pharmacol. 2006 September; 28 Suppl B: 1-56. It also increases the plasma concentration of critical modulators of the HPA axis, which includes adrenocorticotropin (ACTH), serum growth hormone (GH), thyroid-stimulating hormone (TSH), follicle-stimulating hormone (FSH), and luteinizing hormone (LH) levels. See, e.g., Cavun S, Savci V. Fundam Clin Pharmacol. 2004 October; 18 (5): 513-23. In some aspects, the citicoline is selected from Cebroton, Ceraxon, Cognizin, Hipercol or Synapsine.

In certain aspects, the compositions of the present disclosure comprise L-theanine in the amount of about 100 mg, about 105 mg, about 110 mg, about 115 mg, about 120 mg, about 125 mg, about 130 mg, about 135 mg, about 140 mg, about 145 mg, about 150 mg, about 155 mg, 160 mg, about 165 mg, about 170 mg, about 175 mg, about 180 mg, about 185 mg, about 190 mg, about 195 mg, about 200 mg. In certain aspects, the compositions of the present disclosure comprise about 100 mg of L-theanine. In certain aspects, the compositions of the present disclosure comprise 100 mg of L-theanine. In certain aspects, the compositions of the present disclosure comprise about 150 mg of L-theanine. In certain aspects, the compositions of the present disclosure comprise 150 mg of L-theanine. In certain aspects, the compositions of the present disclosure comprise about 175 mg of L-theanine. In certain aspects, the compositions of the present disclosure comprise 175 mg of L-theanine. In certain aspects, the compositions of the present disclosure comprise about 200 mg of L-theanine. In certain aspects, the compositions of the present disclosure comprise 200 mg of L-theanine.

L-theanine, also known as L-γ-glutamylethylamide and N5-ethyl-L-glutamine, is an amino acid analog of the proteinogenic amino acids L-glutamate and L-glutamine and is substantially present in black, green, and white teas. See, e.g., Finger A, Kuhr S, Engelhardt U H. J Chromatogr. 1992 Oct. 30; 624 (1-2): 293-315. L-theanine increases the serotonin and/or dopamine concentrations in the brain. See, e.g., Yokogoshi H et al., Neurochem Res. 1998 May; 23 (5): 667-73.

In some aspects, the compositions of the disclosure comprise an effective amount of Panax quinquefolius, citicoline, and L-theanine. In some aspects, the compositions of the disclosure comprise about 100 mg to about 300 mg of Panax quinquefolius, about 150 mg to about 400 mg of citicoline, and about 100 mg to about 200 mg of L-theanine. In particular aspects, the compositions of the disclosure comprise 150 mg of Panax quinquefolius, 250 mg of citicoline, and 150 mg of L-theanine.

In some aspects, compositions of the present disclosure comprise an effective amount of Panax ginseng, L-theanine, acetyl-L-carnitine, or any combination thereof. In certain aspects, the compositions of the present disclosure comprise about 100 mg to about 400 mg of acetyl-L-carnitine, about 100 mg to about 400 mg of citicoline, about 100 mg to about 300 mg of L-theanine, about 40 mg to about 300 mg of Panax ginseng, or any combination thereof.

In some aspects, the compositions of the present disclosure comprise about 100 mg to about 400 mg of acetyl-L-carnitine, about 40 mg to about 300 mg of Panax quinquefolius or about 40 mg to about 300 mg of Panax ginseng, and about 5 mg to about 200 mg of Sceletium tortuosum. In some aspects, the compositions of the present disclosure comprise about 100 mg to about 400 mg of acetyl-L-carnitine, about 40 mg to about 300 mg of Panax ginseng, and about 5 mg to about 200 mg of Sceletium tortuosum.

In some aspects, the compositions of the present disclosure comprise about 40 mg to about 300 mg of Panax quinquefolius or 40 mg to about 300 mg of Panax ginseng, and about 5 mg to about 200 mg of Sceletium tortuosum

In some aspects, the compositions of the present disclosure comprise about 100 mg to about 400 mg of citicoline about 100 mg to about 300 mg of L-theanine, about 40 mg to about 300 mg of Panax quinquefolius, or about 40 mg to about 300 mg of Panax ginseng, or any combination thereof.

In some aspects, the compositions of the present disclosure comprise about 100 mg to about 300 mg of L-theanine, about 40 mg to about 300 mg of Panax quinquefolius, or about 40 mg to about 300 mg of Panax ginseng, or any combination thereof.

In some aspects, the compositions of the present disclosure comprise an effective amount of acetyl-L-carnitine, citicoline, L-theanine, Panax quinquefolius, Panax ginseng, or any combination thereof. In particular aspects, compositions comprise an effective amount of acetyl-L-carnitine, citicoline, L-Theanine, and Panax quinquefolius. In certain aspects, the compositions of the present disclosure comprise about 100 mg to about 400 mg of acetyl-L-carnitine, about 100 mg to about 400 mg of citicoline, and about 100 mg to about 300 mg of L-theanine, about 40 mg to about 300 mg of Panax quinquefolius, or about 40 mg to about 300 mg of Panax ginseng.

In certain aspects, the compositions of the present disclosure comprise 300 mg acetyl-L-carnitine, 250 mg citicoline, 150 mg L-theanine, and 85 mg Panax quinquefolius. In certain aspects, the compositions of the present disclosure comprise 300 mg acetyl-L-carnitine, 250 mg citicoline, 150 mg L-theanine, and 85 mg Panax ginseng.

In some aspects, the compositions of the disclosure further comprise hemp oil derived from a Cannabis plant. The oils can be derived from different parts of the plant including but not limited to hemp stalk, hemp leaf, hemp flower, and hemp seed. In certain aspects, the compositions of the present disclosure comprise hemp oil in the amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg. In certain aspects, the compositions of the present disclosure comprise about 1 mg of hemp oil. In certain aspects, the compositions of the present disclosure comprise 1 mg of hemp oil. In certain aspects, the compositions of the present disclosure comprise about 2 mg of hemp oil. In certain aspects, the compositions of the present disclosure comprise 2 mg of hemp oil. In certain aspects, the compositions of the present disclosure comprise about 5 mg of hemp oil. In certain aspects, the compositions of the present disclosure comprise 5 mg of hemp oil. In certain aspects, the compositions of the present disclosure comprise about 10 mg of hemp oil. In certain aspects, the compositions of the present disclosure comprise 10 mg of hemp oil.

In some aspects, the compositions according to the present disclosure further comprise one or more cannabinoids. Cannabinoids are compounds found in Cannabis plants that target various aspects of the endocannabinoid system. Notable cannabinoids include cannabidiol (CBD), cannabinol (CBN), and tetrahydrocannabinol (THC)—the compound that produces the euphoric “high” from cannabis consumption. Cannabinoid consumption can have positive effects on, e.g., sleep, anxiety, mood, creativity, focus, and energy levels.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the compositions according to the present disclosure further comprise one or more flavorants. Flavorants are natural or artificial compounds that impart a pleasant flavor and odor to oral formulations. Exemplary flavorants include artificial, natural, and synthetic flavors such as vanilla, chocolate, pumpkin pie, cookie dough, “baked pear” spices, and citrus oils including lemon, orange, lime, grapefruit, yuzu, sudachi, and fruit essences including apple, green apple, pear, peach, grape, berry, blueberry, strawberry, wild berry, date, passion fruit, plum, pineapple, apricot, banana, melon, apricot, cherry, raspberry, blackberry, tropical fruit, lychee, mango, mangosteen, pomegranate, and papaya.

### Compositions for Increasing Stamina and Motivation

The present disclosure also provides compositions for increasing stamina and motivation. Stamina is defined as the strength and vitality required for sustained physical or mental activity. Motivation refers to the ability or willingness to engage in cognitive work. It involves cognition, motivation to do mental work, and feelings of energy and/or absence of feelings of fatigue. Stamina refers to the body's capacity to perform work. See, e.g., Chapelot D, Charlot K. Metabolism. 2019 March; 92:11-25. Energy homeostasis is, in part, regulated by the HPA axis, for example, corticosteroid mediates insulin release and these two hormones have reciprocal effects on energy metabolism. See, e.g., Jéquier E. Ann N Y Acad Sci. 2002 June; 967:379-88; Dallman M F, et al., Ann N Y Acad Sci. 1995 Dec. 29; 771:730-42.

In some aspects, the compositions of the disclosure increase motivation. In some aspects, the compositions of the disclosure increase stamina. In some aspects, the compositions of the disclosure increase motivation and stamina.

In some aspects, the compositions of the present disclosure alleviate adverse effects of cannabis consumption in a subject. Cannabis has been known to increase fatigue, and decrease arousal, vigor, and elation in some subjects. Furthermore, some subjects do not experience strain-specific effects due to the adverse effects. For example, one may experience fatigue despite consuming an energy-boosting Sativa based cannabis product. See, e.g., van Wel J H, et al. J Psychopharmacol. March 2015; 29 (3): 324-34. Thus, the compositions of the present disclosure can offset these issues using adaptogen formulations based on their network pharmacology and predicted effects on these brain regions through regulation of the biological processes involved in the production of energy.

In some aspects, compositions of the present disclosure for increasing motivation and stamina comprise an effective amount of Paullinia cupana, Sceletium tortuosum, Rhodiola rosea, Withania somnifera, Schisandra chinensis, Ilex paraguariensis, or any combination thereof. In certain aspects, the compositions of the present disclosure comprise about 90 mg to about 270 mg of Paullinia cupana, about 5 mg to about 200 mg of Sceletium tortuosum, about 100 mg to about 400 mg of Rhodiola rosea, about 200 mg to about 700 mg of Withania somnifera, about 250 mg to about 400 mg of Schisandra chinensis, and about 100 mg to about 250 mg of Ilex paraguariensis, or any combination thereof.

In some aspects, the compositions of the present disclosure comprise about 90 mg to about 270 mg of Paullinia cupana, about 5 mg to about 200 mg of Sceletium tortuosum, and about 100 mg to about 400 mg of Rhodiola rosea.

In some aspects, compositions of the present disclosure comprise an effective amount of Rhodiola rosea, Paullinia cupana, Sceletium tortuosum, or any combination thereof. In certain aspects, the compositions of the present disclosure comprise about 100 mg to about 400 mg of Rhodiola rosea, about 90 mg to about 270 mg of Paullinia cupana, and about 5 mg to about 200 mg of Sceletium tortuosum, or any combination thereof.

In certain aspects, the compositions of the present disclosure comprise Paullinia cupana, in the amount of about 90 mg, about 100 mg, about 110 mg, about 120 mg, about 130 mg, about 140 mg, about 150 mg, 160 mg, about 170 mg, about 180 mg, about 190 mg, about 200 mg, about 210 mg, about 220 mg, about 230 mg, about 240 mg, about 250 mg, 260 mg, or about 270 mg. In certain aspects, the compositions of the present disclosure comprise about 90 mg of Paullinia cupana. In certain aspects, the compositions of the present disclosure comprise 90 mg of Paullinia cupana. In certain aspects, the compositions of the present disclosure comprise about 150 mg of Paullinia cupana. In certain aspects, the compositions of the present disclosure comprise 150 mg of Paullinia cupana. In certain aspects, the compositions of the present disclosure comprise about 200 mg of Paullinia cupana. In certain aspects, the compositions of the present disclosure comprise 200 mg of Paullinia cupana. In certain aspects, the compositions of the present disclosure comprise about 270 mg of Paullinia cupana. In certain aspects, the compositions of the present disclosure comprise 270 mg of Paullinia cupana.

Paullinia is a genus of flowering shrubs, small trees, and lianas in the soapberry family, Sapindaceae, native to tropical South America, Central America, and the Caribbean. See, e.g., Sapindaceae, Meyler's Side Effects of Drugs (Sixteenth Edition), Elsevier, 2016, Page 305.

Paullinia species of the present disclosure include, but are not limited to, Paullinia cupana, Paullinia alata, Paullinia cupana Kunth-Guaraná (Amazon Basin), Paullinia cururu, Paullinia fuscescens, Paullinia navicularis Radlk. (Ecuador), Paullinia paullinioides, Paullinia pinnata, Paullinia plumieri, Paullinia weinmannifolia and Paullinia yoco—Yoco.

Paullinia cupana, better known as Guaraná, contains guaraná and is identical to caffeine derived from other sources. It contains methylxanthine alkaloids and phenols: (+)-catechin and (−)-epicatechin. See, e.g., “Caffeine”. Biological Magnetic Resonance Data Bank, University of Wisconsin-Madison. Archived from the original on 2007 Nov. 24; Carlson M, Thompson R D. J AOAC Int. 1998 July-August; 81 (4): 691-701. The pharmacological activity of guaraná-containing products is primarily due to methylxanthine alkaloids.

In certain aspects, the compositions of the present disclosure comprise Sceletium tortuosum in the amount of about 5 mg, about 10 mg, about 15 mg, about 20 mg, about 25 mg, about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, 160 mg, about 165 mg, about 170 mg, about 175 mg, about 180 mg, about 185 mg, about 190 mg, about 195 mg, about 200 mg. In certain aspects, the compositions of the present disclosure comprise about 50 mg of Sceletium tortuosum. In certain aspects, the compositions of the present disclosure comprise 50 mg of Sceletium tortuosum. In certain aspects, the compositions of the present disclosure comprise about 75 mg of Sceletium tortuosum. In certain aspects, the compositions of the present disclosure comprise 75 mg of Sceletium tortuosum. In certain aspects, the compositions of the present disclosure comprise about 100 mg of Sceletium tortuosum. In certain aspects, the compositions of the present disclosure comprise 100 mg of Sceletium tortuosum. In certain aspects, the compositions of the present disclosure comprise about 200 mg of Sceletium tortuosum. In certain aspects, the compositions of the present disclosure comprise 200 mg of Sceletium tortuosum.

In certain aspects, the compositions of the present disclosure comprise Rhodiola rosea, in the amount of about 100 mg, about 110 mg, about 120 mg, 130 mg, about 140 mg, about 150 mg, about 160 mg, about 170 mg, about 180 mg, about 190 mg, 200 mg, about 210 mg, about 220 mg, 230 mg, about 240 mg, about 250 mg, about 260 mg, about 270 mg, about 280 mg, about 290 mg, about 300 mg, about 310 mg, about 320 mg, 330 mg, about 340 mg, about 350 mg, about 360 mg, about 370 mg, about 380 mg, about 390 mg, or about 400 mg. In certain aspects, the compositions of the present disclosure comprise about 100 mg of Rhodiola rosea. In certain aspects, the compositions of the present disclosure comprise 100 mg of Rhodiola rosea. In certain aspects, the compositions of the present disclosure comprise about 150 mg of Rhodiola rosea. In certain aspects, the compositions of the present disclosure comprise 150 mg of Rhodiola rosea. In certain aspects, the compositions of the present disclosure comprise about 200 mg of Rhodiola rosea. In certain aspects, the compositions of the present disclosure comprise 200 mg of Rhodiola rosea. In certain aspects, the compositions of the present disclosure comprise about 300 mg of Rhodiola rosea. In certain aspects, the compositions of the present disclosure comprise 300 mg of Rhodiola rosea.

Rhodiola is a genus of perennial plants in the family Crassulaccac. See, e.g., Stevens, P. F. (2019) [2001]. “Crassulaceae”. Angiosperm Phylogeny Website. Missouri Botanical Garden.

Rhodiola species of the present disclosure include, but are not limited to, Rhodiola integrifolia, Rhodiola crenulata, Rhodiola cretinii, Rhodiola imbricata, Rhodiola kirilowii, Rhodiola rhodantha, Rhodiola rosea, Rhodiola tibetica, and Rhodiola quadrifida.

Rhodiola rosea, commonly golden root, contains over 140 chemical compounds, including phenols, rosavin, terpenoids, and phenolic acids. See, e.g. Evstaticva L, et al. Pharmacogn Mag. 2010; 6 (24): 256-258. It influences the levels of monoamines, including serotonin, dopamine, and norepinephrine, through inhibition of degradation enzymes and facilitation of neurotransmitter support in the brain. See, e.g., Mary Bove, Jillian E. Stansbury, Aviva Romm. CHAPTER 6-Endocrine Disorders and Adrenal Support, Botanical Medicine for Women's Health. 2010, Pages 186-210, ISBN 9780443072772.

In some aspects of the disclosure, the composition comprises an effective amount of Paullinia cupana, Sceletium tortuosum, and Rhodiola rosea. In some aspects, the composition comprises about 90 mg to about 270 mg of Paullinia cupana, about 5 mg to about 200 mg of Sceletium tortuosum, and about 100 mg to about 400 mg of Rhodiola rosea. In particular aspects, the composition comprises 160 mg of Paullinia cupana, 35 mg of Sceletium tortuosum, and 250 mg of Rhodiola rosea. In particular aspects, the composition comprises 180 mg of Paullinia cupana, 35 mg of Sceletium tortuosum, and 250 mg of Rhodiola rosea.

In some aspects, the compositions of the disclosure further comprise hemp oil derived from a Cannabis plant. The oils can be derived from different parts of the plant including but not limited to hemp stalk, hemp leaf, hemp flower, and hemp seed. In certain aspects, the compositions of the present disclosure comprise hemp oil in the amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg. In certain aspects, the compositions of the present disclosure comprise about 1 mg of hemp oil. In certain aspects, the compositions of the present disclosure comprise 1 mg of hemp oil. In certain aspects, the compositions of the present disclosure comprise about 2 mg of hemp oil. In certain aspects, the compositions of the present disclosure comprise 2 mg of hemp oil. In certain aspects, the compositions of the present disclosure comprise about 5 mg of hemp oil. In certain aspects, the compositions of the present disclosure comprise 5 mg of hemp oil. In certain aspects, the compositions of the present disclosure comprise about 10 mg of hemp oil. In certain aspects, the compositions of the present disclosure comprise 10 mg of hemp oil.

In some aspects, the compositions according to the present disclosure further comprise one or more cannabinoids. Cannabinoids are compounds found in Cannabis plants that target various aspects of the endocannabinoid system. Notable cannabinoids include cannabidiol (CBD), cannabinol (CBN), and tetrahydrocannabinol (THC)—the compound that produces the euphoric “high” from cannabis consumption. Cannabinoid consumption can have positive effects on, e.g., sleep, anxiety, mood, creativity, focus, and energy levels.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the compositions according to the present disclosure further comprise one or more flavorants. Flavorants are natural or artificial compounds that impart a pleasant flavor and odor to oral formulations. Exemplary flavorants include artificial, natural, and synthetic flavors such as vanilla, chocolate, pumpkin pie, cookie dough, “baked pear” spices, and citrus oils including lemon, orange, lime, grapefruit, yuzu, sudachi, and fruit essences including apple, green apple, pear, peach, grape, berry, blueberry, strawberry, wild berry, date, passion fruit, plum, pineapple, apricot, banana, melon, apricot, cherry, raspberry, blackberry, tropical fruit, lychee, mango, mangosteen, pomegranate, and papaya.

### Compositions for Increasing Sociability

In some aspects the compositions of the present disclosure for increasing sociability comprise an effective amount of Withania somnifera, Schisandra chinensis, Ilex paraguariensis, or any combination thereof. In some aspects, the compositions of the disclosure comprise an effective amount of Withania somnifera, Schisandra chinensis, Ilex paraguariensis and Griffonia simplicifolia. In some aspects, the compositions of the present disclosure comprise about 200 mg to about 700 mg of Withania somnifera, about 150 mg to about 400 mg of Schisandra chinensis, about 100 mg to about 250 mg of Ilex paraguariensis and about 20 mg to about 200 mg of Griffonia simplicifolia.

In certain aspects, the compositions of the present disclosure comprise Withania somnifera in the amount of about 200 mg, about 210 mg, about 220 mg, 230 mg, about 240 mg, about 250 mg, about 260 mg, about 270 mg, about 280 mg, about 290 mg, about 300 mg, about 310 mg, about 320 mg, 330 mg, about 340 mg, about 350 mg, about 360 mg, about 370 mg, about 380 mg, about 390 mg, about 400 mg, about 410 mg, about 420 mg, 430 mg, about 440 mg, about 450 mg, about 460 mg, about 470 mg, about 480 mg, about 490 mg, about 500 mg, about 510 mg, about 520 mg, 530 mg, about 540 mg, about 550 mg, about 560 mg, about 570 mg, about 580 mg, about 590 mg, about 600 mg, about 610 mg, about 620 mg, 630 mg, about 640 mg, about 650 mg, about 660 mg, about 670 mg, about 680 mg, about 690 mg, or about 700 mg. In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 300 mg of Withania somnifera. In certain aspects, the compositions of the present disclosure comprise 300 mg of Withania somnifera. In certain aspects, the compositions of the present disclosure comprise about 400 mg of Withania somnifera. In certain aspects, the compositions of the present disclosure comprise 400 mg of Withania somnifera. In certain aspects, the compositions of the present disclosure comprise about 500 mg of Withania somnifera. In certain aspects, the compositions of the present disclosure comprise 500 mg of Withania somnifera. In certain aspects, the compositions of the present disclosure comprise about 600 mg of Withania somnifera. In certain aspects, the compositions of the present disclosure comprise 600 mg of Withania somnifera. In certain aspects, the compositions of the present disclosure comprise about 700 mg of Withania somnifera. In certain aspects, the compositions of the present disclosure comprise 700 mg of Withania somnifera.

Withania is a genus of flowering plants in the nightshade family, Solanaceae, with about 23 species native to North Africa, Western Asia, South Asia, southern Europe, the Mediterranean, and the Canary Islands. See, e.g., Mirjalili, M. H. et al., Molecules, 14 (7): 2373-2393 (2009); “Withania somnifera.” Germplasm Resources Information Network (GRIN). Agricultural Research Service (ARS), United States Department of Agriculture (USDA). Accessed 28 Apr. 2020.

Withania species of the present disclosure include, but are not limited to, Withania somnifera (Ashwaganda, Indian ginseng, winter cherry), Withania adpressa, Withania adunensis, Withania begoniifolia, Withania chevalier, Withania coagulans, Withania frutescens, Withania japonica, Withania qaraitica, Withania reichenbachii, Withania riebeckii, and Withania sphaerocarpa.

In certain aspects, the compositions of the present disclosure comprise Schisandra chinensis, in the amount of about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg. In certain aspects, the compositions of the present disclosure comprise about 250 mg of Schisandra chinensis. In certain aspects, the compositions of the present disclosure comprise 250 mg of Schisandra chinensis. In certain aspects, the compositions of the present disclosure comprise about 275 mg of Schisandra chinensis. In certain aspects, the compositions of the present disclosure comprise 275 mg of Schisandra chinensis. In certain aspects, the compositions of the present disclosure comprise about 300 mg of Schisandra chinensis. In certain aspects, the compositions of the present disclosure comprise 300 mg of Schisandra chinensis. In certain aspects, the compositions of the present disclosure comprise about 350 mg of Schisandra chinensis. In certain aspects, the compositions of the present disclosure comprise 350 mg of Schisandra chinensis. In certain aspects, the compositions of the present disclosure comprise about 375 mg of Schisandra chinensis. In certain aspects, the compositions of the present disclosure comprise 375 mg of Schisandra chinensis. In certain aspects, the compositions of the present disclosure comprise about 400 mg of Schisandra chinensis. In certain aspects, the compositions of the present disclosure comprise 400 mg of Schisandra chinensis.

Schisandra (magnolia vine) is a genus of twining shrub in family Schisandraceae, native to Asia and North America, with a center of diversity in China. See, e.g., Hutchinson, J., ed. 3. Oxford. pp. 161-162 (1973); Smith, A. C. Sargentia 7:1-224 (1947).

Schisandra species of the present disclosure include, but are not limited to, Schisandra chinensis (magnolia-vine, Chinese magnolia-vine, schisandra, whose fruit is called magnolia berry or five-flavor-fruit; see e.g., Nowak A., et al., Nutrients., 11 (2): 333 (2019).), Schisandra arisanensis, Schisandra bicolor, Schisandra elongata, Schisandra glabra, Schisandra glaucescens, Schisandra grandiflora, Schisandra henryi, Schisandra incarnata, Schisandra lancifolia, Schisandra macrocarpa, Schisandra micrantha, Schisandra neglecta, Schisandra parapropinqua, Schisandra perulata, Schisandra plena, Schisandra propinqua, Schisandra pubescens, Schisandra pubinervis, Schisandra repanda, Schisandra rubriflora, Schisandra sphaerandra, Schisandra sphenanthera, and Schisandra tomentella.

Schisandra chinensis contains many active compounds, including triterpenoids, dibenzo[a, c] cyclooctadiene lignans, and polyphenols. See, e.g., Szopa, Agnieszka et al. Phytochemistry reviews: proceedings of the Phytochemical Society of Europe vol. 16, 2 (2017): 195-218. Schisandra chinensis inhibits acetylcholinesterase activity, regulates CREB-mediated Bcl-2 expression, and activates PI3K/Akt survival signaling. Jeong E J, et al. J Ethnopharmacol. 2013 Mar. 7; 146 (1): 347-54; Sa F, et al., Neurosci Lett. 2015 Apr. 23; 593:7-12.

In certain aspects, the compositions of the present disclosure comprise Ilex paraguariensis, in the amount of about 100 mg, about 110 mg, about 120 mg, 130 mg, about 140 mg, about 150 mg, about 160 mg, about 170 mg, about 180 mg, about 190 mg, 200 mg, about 210 mg, about 220 mg, 230 mg, about 240 mg, or about 250 mg. In certain aspects, the compositions of the present disclosure comprise about 100 mg of Ilex paraguariensis. In certain aspects, the compositions of the present disclosure comprise 100 mg of Ilex paraguariensis. In certain aspects, the compositions of the present disclosure comprise about 150 mg of Ilex paraguariensis. In certain aspects, the compositions of the present disclosure comprise 150 mg of Ilex paraguariensis. In certain aspects, the compositions of the present disclosure comprise about 200 mg of Ilex paraguariensis. In certain aspects, the compositions of the present disclosure comprise 200 mg of Ilex paraguariensis. In certain aspects, the compositions of the present disclosure comprise about 250 mg of Ilex paraguariensis. In certain aspects, the compositions of the present disclosure comprise 250 mg of Ilex paraguariensis.

Ilex, or holly, is a genus of over 560 species of flowering plants in the family Aquifoliaceae and is widespread throughout the temperate and subtropical regions of the world. See, e.g., Sunset Western Garden Book. 1995. pp. 606-07.

Ilex species of the present disclosure include, but are not limited to, Ilex paraguariensis, Ilex ambigua, Ilex anomala, Ilex guayusa, Ilex integra, Ilex mucronata, and Ilex opaca.

Ilex paraguariensis, better known as Yerba-mate, contains various polyphenols such as the flavonoids quercetin and rutin. See, e.g., Gambero A, Ribeiro M L. Nutrients. 2015 Jan. 22; 7 (2): 730-50. It increases antioxidant defenses and improves liver metabolism through improvements in hypothalamic activity. See, e.g., Conceição E P, et al. J Dev Orig Health Dis. 2017 February; 8 (1): 123-132.

In certain aspects, the compositions of the present disclosure comprise Griffonia simplicifolia in the amount of about 5 mg, about 10 mg, about 15 mg, about 20 mg, about 25 mg, about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, or about 100 mg. In certain aspects, the compositions of the present disclosure comprise about 20 mg of Griffonia simplicifolia. In certain aspects, the compositions of the present disclosure comprise 20 mg of Griffonia simplicifolia. In certain aspects, the compositions of the present disclosure comprise about 40 mg of Griffonia simplicifolia. In certain aspects, the compositions of the present disclosure comprise 40 mg of Griffonia simplicifolia. In certain aspects, the compositions of the present disclosure comprise about 80 mg of Griffonia simplicifolia. In certain aspects, the compositions of the present disclosure comprise 80 mg of Griffonia simplicifolia. In certain aspects, the compositions of the present disclosure comprise about 100 mg of Griffonia simplicifolia. In certain aspects, the compositions of the present disclosure comprise 100 mg of Griffonia simplicifolia.

Griffonia is a genus of flowering plants in the legume family, Fabaceae. It belongs to the subfamily Cercidoideae. Griffonia species of the present disclosure include, but are not limited to Griffonia physocarpa, Griffonia speciose, Griffonia tessmannii and Griffonia simplicifolia. See, e.g., Baillon, H E. Adansonia 6:188. 1865.

Griffonia simplicifolia seeds produce a high concentration of 5-hydroxytryptophan (5-HTP). 5-HTP is an important building block for the human body to form serotonin, a neurotransmitter and thus Griffonia simplicifolia increases the concentration of serotonin. See, e.g., Lemaire P A, Adosraku R K. Phytochem Anal. 2002 November-December; 13 (6): 333-7.

In some aspects of the disclosure, the composition comprises an effective amount of Withania somnifera, Schisandra chinensis, Ilex paraguariensis, or any combination thereof. In some aspects of the disclosure, the composition comprises an effective amount of Withania somnifera, Schisandra chinensis, and Ilex paraguariensis. In some aspects, the compositions of the present disclosure comprise about 200 mg to about 700 mg of Withania somnifera, about 250 mg to about 400 mg of Schisandra chinensis, and about 100 mg to about 250 mg of Ilex paraguariensis. In particular aspects, the composition comprises 320 mg of Withania somnifera, 320 mg of Schisandra chinensis, and 160 mg of Ilex paraguariensis.

In some aspects of the disclosure, the composition comprises an effective amount of Withania somnifera, Schisandra chinensis, Ilex paraguariensis, Griffonia simplicifolia, or any combination thereof. In some aspects of the disclosure, the composition comprises an effective amount of Withania somnifera, Schisandra chinensis, Ilex paraguariensis, and Griffonia simplicifolia. In some aspects, the compositions of the present disclosure comprise about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia. In particular aspects, the composition comprises 320 mg of Withania somnifera, 220 mg of Schisandra chinensis, 180 mg of Ilex paraguariensis, and 100 mg of Griffonia simplicifolia. In particular aspects, the composition comprises 318 mg of Withania somnifera, 220 mg of Schisandra chinensis, 160 mg of Ilex paraguariensis, and 100 mg of Griffonia simplicifolia.

In some aspects, the compositions of the disclosure further comprise hemp oil derived from a Cannabis plant. The oils can be derived from different parts of the plant including but not limited to hemp stalk, hemp leaf, hemp flower, and hemp seed. In certain aspects, the compositions of the present disclosure comprise hemp oil in the amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg. In certain aspects, the compositions of the present disclosure comprise about 1 mg of hemp oil. In certain aspects, the compositions of the present disclosure comprise 1 mg of hemp oil. In certain aspects, the compositions of the present disclosure comprise about 2 mg of hemp oil. In certain aspects, the compositions of the present disclosure comprise 2 mg of hemp oil. In certain aspects, the compositions of the present disclosure comprise about 5 mg of hemp oil. In certain aspects, the compositions of the present disclosure comprise 5 mg of hemp oil. In certain aspects, the compositions of the present disclosure comprise about 10 mg of hemp oil. In certain aspects, the compositions of the present disclosure comprise 10 mg of hemp oil.

In some aspects, the compositions according to the present disclosure further comprise one or more cannabinoids. Cannabinoids are compounds found in Cannabis plants that target various aspects of the endocannabinoid system. Notable cannabinoids include cannabidiol (CBD), cannabinol (CBN), and tetrahydrocannabinol (THC)—the compound that produces the euphoric “high” from cannabis consumption. Cannabinoid consumption can have positive effects on, e.g., sleep, anxiety, mood, creativity, focus, and energy levels.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the compositions according to the present disclosure further comprise one or more flavorants. Flavorants are natural or artificial compounds that impart a pleasant flavor and odor to oral formulations. Exemplary flavorants include artificial, natural, and synthetic flavors such as vanilla, chocolate, pumpkin pie, cookie dough, “baked pear” spices, and citrus oils including lemon, orange, lime, grapefruit, yuzu, sudachi, and fruit essences including apple, green apple, pear, peach, grape, berry, blueberry, strawberry, wild berry, date, passion fruit, plum, pineapple, apricot, banana, melon, apricot, cherry, raspberry, blackberry, tropical fruit, lychee, mango, mangosteen, pomegranate, and papaya.

Compositions for Recovery from Stress

The present disclosure also provides compositions for recovery from stress. Stress is a biological reaction to a potentially dangerous situation, and the two main forms are physiological and psychosocial stress. Psychological stress, the emotion and mental strain, precedes physiological stress which is the body's response. Both are managed by the endocrine system, the first is the norepinephrine sympathetic adrenal medullary system, which regulates blood pressure, heart rate and release of catecholamines, and the second is the HPA-axis, which regulates release of corticosteroids. See, e.g., Andrew Steptoe, Lena Brydon. Psychoneuroimmunology (Fourth Edition), 2007. Pages 945-974, ISBN 9780120885763; Andrew Steptoe. Comprehensive Clinical Psychology, 1998. Pages 39-78, ISBN 9780080427072; Kogler L, Müller VI, Chang A, et al. Neuroimage. 2015; 119:235-251.

In some aspects, the compositions of the disclosure reduce psychological stress, physiological stress, pain, or any combination thereof. In some aspects, the compositions of the present disclosure reduce stress. In some aspects, the compositions of the disclosure reduce psychological stress. In some aspects, the composition of the present disclosure reduce physiological stress. In some aspects, the compositions of the present disclosure reduce pain. In some aspects, the compositions of the disclosure reduce psychological stress, physiological stress, and pain.

In some aspects, the compositions of the disclosure reduce the effects of psychological stress, physiological stress, pain, or any combination thereof. In some aspects, the compositions of the disclosure enhance recovery from psychological stress. In some aspects, the composition of the present disclosure enhance recovery from physiological stress. In some aspects, the compositions of the present disclosure reduce pain. In some aspects, the compositions of the disclosure reduce the negative effects of psychological stress, as well as reduce pain.

In some aspects, the compositions of the present disclosure alleviate the adverse effects of cannabis. Cannabis has been known to increase depression and anxiety and decreased positive mood, friendliness in some subjects. Thus, some subjects do not experience the stress-relieving effects of Cannabis and the compositions of the present disclosure alleviates these adverse effects. The compositions of the present disclosure can offset these issues using adaptogen formulations based on their network pharmacology and predicted effects on these brain regions through regulation of the multiple pathways involved in the stress response resulting in decreased anxiety, improved mood, etc. In certain aspects, the compositions of the disclosure provide anti-inflammatory benefits which when combined with the analgesic properties of cannabis products results in reduction of pain and physiological stress response.

In some aspects of the disclosure, the compositions for promoting relaxation and relieving stress comprise an effective amount of Withania somnifera, Melissa officinalis essential oil, Melissa officinalis, Passiflora incarnata, Piper methysticum, Griffonia simplicifolia, Corydalis yanhusuo, or any combination thereof.

In certain aspects, the compositions of the present disclosure comprise about 200 mg to about 700 mg of Withania somnifera, about 0.1 mg to about 1 mg of Melissa officinalis essential oil, about 50 mg to about 150 mg of Melissa officinalis, about 50 mg to about 300 mg of Passiflora incarnata, about 25 mg to about 100 mg of Piper methysticum, 100 mg to about 300 mg of Corydalis yanhusuo, or any combination thereof. In other aspects, the compositions of the present disclosure comprise about 200 mg to about 700 mg of Withania somnifera, about 0.1 mg to about 1 mg of Melissa officinalis essential oil, about 50 mg to about 150 mg of Melissa officinalis, about 50 mg to about 300 mg of Passiflora incarnata, about 25 mg to about 100 mg of Piper methysticum, about 5 mg to about 100 mg of Griffonia simplicifolia, and 100 mg to about 300 mg of Corydalis yanhusuo.

In some aspects, the compositions of the present disclosure comprise about 0.1 mg to about 1 mg of Melissa officinalis essential oil, about 50 mg to about 150 mg of Melissa officinalis, about 200 mg to about 700 mg of Withania somnifera, and about 50 mg to about 300 mg of Passiflora incarnata.

In some aspects of the disclosure, the compositions of the present disclosure comprise an effective amount of Withania somnifera, Corydalis yanhusuo, Silybum marianum Melissa officinalis, Passiflora incarnata, Piper methysticum, Griffonia simplicifolia, or any combination thereof.

In certain aspects, the compositions of the present disclosure comprise about 200 mg to about 700 mg of Withania somnifera, about 50 mg to about 150 mg of Melissa officinalis, about 50 mg to about 300 mg of Passiflora incarnata, about 25 mg to about 100 mg of Piper methysticum, 100 mg to about 300 mg of Corydalis yanhusuo, about 30 mg to about 140 mg of Silybum marianum, and about 20 mg to about 200 mg of Griffonia simplicifolia, or any combination thereof. In other aspects, the compositions of the present disclosure comprise about 200 mg to about 700 mg of Withania somnifera, about 50 mg to about 150 mg of Melissa officinalis, about 50 mg to about 300 mg of Passiflora incarnata, about 25 mg to about 100 mg of Piper methysticum, about 5 mg to about 100 mg of Griffonia simplicifolia, and 100 mg to about 300 mg of Corydalis yanhusuo, 80 mg of Silybum marianum, about 40 mg of Griffonia simplicifolia, or any combination thereof.

In some aspects, the compositions of the present disclosure comprise about 50 mg to about 150 mg of Melissa officinalis, about 200 mg to about 700 mg of Withania somnifera, and about 50 mg to about 300 mg of Passiflora incarnata.

In some aspects, the compositions of the present disclosure comprise about 200 mg to about 700 mg of Withania somnifera, about 100 mg to about 300 mg of Corydalis yanhusuo, about 30 mg to about 140 mg of Silybum marianum, and about 5 mg to about 100 mg of Griffonia simplicifolia. In some aspects, the compositions of the present disclosure comprise about 480 mg of Withania somnifera, about 200 of Corydalis yanhusuo, about 80 mg of Silybum marianum, and about 40 mg of Griffonia simplicifolia. In some aspects, the compositions of the present disclosure comprise 478 mg of Withania somnifera, 200 of Corydalis yanhusuo, 80 mg of Silybum marianum, and 40 mg of Griffonia simplicifolia.

In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 300 mg of Withania somnifera. In certain aspects, the compositions of the present disclosure comprise 300 mg of Withania somnifera. In certain aspects, the compositions of the present disclosure comprise about 400 mg of Withania somnifera. In certain aspects, the compositions of the present disclosure comprise 400 mg of Withania somnifera. In certain aspects, the compositions of the present disclosure comprise about 500 mg of Withania somnifera. In certain aspects, the compositions of the present disclosure comprise 500 mg of Withania somnifera. In certain aspects, the compositions of the present disclosure comprise about 600 mg of Withania somnifera. In certain aspects, the compositions of the present disclosure comprise 600 mg of Withania somnifera. In certain aspects, the compositions of the present disclosure comprise about 700 mg of Withania somnifera. In certain aspects, the compositions of the present disclosure comprise 700 mg of Withania somnifera.

In certain aspects, the compositions of the present disclosure comprise Silybum marianum in the amount of about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, or about 140 mg. In certain aspects, the compositions of the present disclosure comprise about 40 mg of Silybum marianum. In certain aspects, the compositions of the present disclosure comprise 60 mg of Silybum marianum. In certain aspects, the compositions of the present disclosure comprise about 80 mg of Silybum marianum. In certain aspects, the compositions of the present disclosure comprise 80 mg of Silybum marianum. In certain aspects, the compositions of the present disclosure comprise about 100 mg of Silybum marianum. In certain aspects, the compositions of the present disclosure comprise 100 mg of Silybum marianum.

Silybum (milk thistle) is a genus of two species of thistles in the family Asteraceae. As for Silybum marianum (Milk thistle), it is a plant native to Southern Europe and Asia. It is known for producing red to purple flowers, shiny pale green leaves with white veins, and fruit.

Silybum marianum extract consists of about 65-80% silymarin (a flavonolignan complex) and 20-35% fatty acids, including linoleic acid. [23] Silymarin is a complex mixture of polyphenolic molecules, including seven closely related flavonolignans (silybin A, silybin B, isosilybin A, isosilybin B, silychristin, isosilychristin, silydianin) and one flavonoid (taxifolin)

In certain aspects, the compositions of the present disclosure comprise Melissa officinalis essential oil in the amount of about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 0.96 mg, about 0.97 mg, about 0.98 mg, or about 1 mg. In certain aspects, the compositions of the present disclosure comprise about 0.1 mg of Melissa officinalis essential oil. In certain aspects, the compositions of the present disclosure comprise 0.1 mg of Melissa officinalis essential oil. In certain aspects, the compositions of the present disclosure comprise about 0.25 mg of Melissa officinalis essential oil. In certain aspects, the compositions of the present disclosure comprise 0.25 mg of Melissa officinalis essential oil. In certain aspects, the compositions of the present disclosure comprise about 0.5 mg of Melissa officinalis essential oil. In certain aspects, the compositions of the present disclosure comprise 0.5 mg of Melissa officinalis essential oil. In certain aspects, the compositions of the present disclosure comprise about 1 mg of Melissa officinalis essential oil. In certain aspects, the compositions of the present disclosure comprise 1 mg of Melissa officinalis essential oil.

In certain aspects, the compositions of the present disclosure comprise Melissa officinalis in the amount of about 50 mg, about 55 mg, 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, or about 150 mg. In certain aspects, the compositions of the present disclosure comprise about 50 mg of Melissa officinalis. In certain aspects, the compositions of the present disclosure comprise 50 mg of Melissa officinalis. In certain aspects, the compositions of the present disclosure comprise about 75 mg of Melissa officinalis. In certain aspects, the compositions of the present disclosure comprise 75 mg of Melissa officinalis. In certain aspects, the compositions of the present disclosure comprise about 100 mg of Melissa officinalis. In certain aspects, the compositions of the present disclosure comprise 100 mg of Melissa officinalis. In certain aspects, the compositions of the present disclosure comprise about 150 mg of Melissa officinalis. In certain aspects, the compositions of the present disclosure comprise 150 mg of Melissa officinalis.

Melissa is a genus of perennial herbs in the family Lamiaceae. Melissa species of the present disclosure include, but are not limited to Melissa officinalis (e.g., Shakeri A. at al., J Ethnopharmacol. 188:204-228 (2016)).

Melissa officinalis contains monoterpenoid aldehydes (including citronellal, neral, and geranial), flavonoids, and polyphenolic compounds such as rosmarinic acid and monoterpene glycosides. It is an anti-oxidant, regulates the cholinergic system, and increases GABA levels in the brain by inhibiting the GABA transaminase enzyme.

In certain aspects, the compositions of the present disclosure comprise Passiflora incarnata in the amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 200 mg. In certain aspects, the compositions of the present disclosure comprise about 50 mg of Passiflora incarnata. In certain aspects, the compositions of the present disclosure comprise 50 mg of Passiflora incarnata. In certain aspects, the compositions of the present disclosure comprise about 100 mg of Passiflora incarnata. In certain aspects, the compositions of the present disclosure comprise 100 mg of Passiflora incarnata. In certain aspects, the compositions of the present disclosure comprise about 150 mg of Passiflora incarnata. In certain aspects, the compositions of the present disclosure comprise 150 mg of Passiflora incarnata. In certain aspects, the compositions of the present disclosure comprise about 200 mg of Passiflora incarnata. In certain aspects, the compositions of the present disclosure comprise 200 mg of Passiflora incarnata. In certain aspects, the compositions of the present disclosure comprise about 300 mg of Passiflora incarnata. In certain aspects, the compositions of the present disclosure comprise 300 mg of Passiflora incarnata.

Passiflora, also known as the passion flowers or passion vines, is a genus of about 550 species of flowering plants, the type genus of the family Passifloraceae. See e.g., Cerqueira-Silva C B. et al., Int J Mol Sci., 15 (8): 14122-14152 (2014).

Passiflora species of the present disclosure include, but are not limited to, Passiflora incarnata (maypops-passionflower, purple passionflower, apricot vine, maypop, wild passionflower, see e.g., Park J W et al., Int J Mol Med., 41 (6): 3709-3716 (2018)), Passiflora actinia (passionflower), Passiflora antioquiensis (banana passionfruit), Passiflora caerulea (passionflower, blue crown passionflower, blue passionflower), Passiflora edulis (passionflower, purple granadilla), Passiflora herbertiana, Passiflora laurifolia (yellow granadilla), Passiflora ligularis (sweet grenadilla, passionflower), Passiflora membranacea (passionflower), Passiflora mollisima (banana passion fruit), Passiflora popenovii (granadilla de quijos), Passiflora quadrangularis (giant granadilla, badea), Passiflora tarminiana (banana passionfruit, banana poka), Passiflora tetrandra, Passiflora tripartita mollissima (banana passionfruit), Passiflora umbilicata (passion flower), Passiflora^{x} colvillii (passion flower), and Passiflora^{x} exoniensis (passion flower).

Passiflora incarnata can activate the GABA-A receptor and 5-hydroxytryptamine (5-HT) receptors, modulate the opioid neuropeptide systems and enhance ERK1/2 activity, inhibit COX-2 expression, and inhibit activation of NF-κB. In certain aspects, the compositions of the present disclosure comprise at least one GABA-A receptor activator. In certain aspects, at least one GABA-A receptor activator is Passiflora incarnata. See, e.g., Appel K. et al., Phytother Res., 25 (6): 838-843 (2011); Park J W, et al., Int J Molec Med, 41 (6): 3709-3716 (2018).

In some aspects of the disclosure, the compositions for promoting relaxation and relieving stress comprise an effective amount of Withania somnifera, Melissa officinalis essential oil, Melissa officinalis, Passiflora incarnata, or any combination thereof. In some aspects of the disclosure, the compositions of the disclosure comprise an effective amount of Withania somnifera, Melissa officinalis essential oil, Melissa officinalis and Passiflora incarnata. In some aspects of the disclosure, the compositions for recovery from stress comprise an effective amount of Withania somnifera, Melissa officinalis, Passiflora incarnata, or any combination thereof. In some aspects of the disclosure, the compositions of the disclosure comprise an effective amount of Withania somnifera, Melissa officinalis and Passiflora incarnata. In some aspects, the compositions of the present disclosure comprise about 0.1 mg to about 1 mg of, about 50 mg to about 150 mg of Melissa officinalis, about 200 mg to about 700 mg of Withania somnifera, and about 50 mg to about 300 mg of Passiflora incarnata.

In particular aspects, the compositions of the disclosure comprise 400 mg of Withania somnifera, 0.5 mg of Melissa officinalis essential oil, 100 mg of Melissa officinalis, and 200 mg of Passiflora incarnata.

In some aspects, the compositions of the disclosure comprise an effective amount of Withania somnifera, and Piper methysticum. In particular aspects, the compositions of the disclosure comprise about 200 mg to about 700 mg of Withania somnifera, and about 25 mg to about 100 mg of Piper methysticum.

In certain aspects, the compositions of the present disclosure comprise Piper methysticum in the amount of about 20 mg, about 25 mg, about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, about 100 mg. In certain aspects, the compositions of the present disclosure comprise about 25 mg of Piper methysticum. In certain aspects, the compositions of the present disclosure comprise 25 mg of Piper methysticum. In certain aspects, the compositions of the present disclosure comprise about 50 mg of Piper methysticum. In certain aspects, the compositions of the present disclosure comprise 50 mg of Piper methysticum. In certain aspects, the compositions of the present disclosure comprise about 75 mg of Piper methysticum. In certain aspects, the compositions of the present disclosure comprise 75 mg of Piper methysticum. In certain aspects, the compositions of the present disclosure comprise about 100 mg of Piper methysticum. In certain aspects, the compositions of the present disclosure comprise 100 mg of Piper methysticum.

Piper is a genus of 1,000-2,000 species in the family Piperaceae. See, e.g., Salehi B et al., Molecules. 7: 24 (7) (2019). Piper species of the present disclosure include, but are not limited to, Piper methysticum, Piper nigrum, Piper abalienatum, Piper abbadianum, Piper abbreviatum, Piper abditum, Piper abellinum, Piper abutilifolium, Piper abutiloides, Piper adamatum, Piper addisonii, Piper adenandrum, Piper aduncum, Piper aequale, Piper aereum, Piper affictum, Piper afflictum, Piper agellifolium, Piper agostiniorum, Piper aguadulcense, Piper alatabaccum, Piper alatipetiolatum, Piper albanense, Piper albert-smithii, Piper albiciliatum, Piper albidum, Piper albogranulatum, Piper albopapillatum, Piper albopilosum, Piper albozonatum, Piper aleyreanum, Piper allardii, Piper alnoides, Piper alveolatum, Piper bahianum, Piper bahiense, Piper bajanum, Piper caballocochanum, Piper cabralanum, Piper dactylostigmum, Piper daedalum, Piper daguanum, Piper ecallosum, Piper echinocaule, Piper factum, Piper faecatum, Piper fagifolium, Piper gabrielianum, Piper galeatum, and Piper galeottianum.

Piper methysticum, better known as Kava, contains 18 kavalactones, including kavain, dihydrokavain, methysticin, dihydromethysticin, yangonin, and desmethoxyyangonin. See, e.g., Sarris J, LaPorte E, Schweitzer I. Aust N Z J Psychiatry. (2011). 45 (1): 27-35. It potentiates GABA-A receptor activity, inhibits norepinephrine and dopamine uptake, binds to the CB1 receptor, and inhibits monoamine oxidases. See, e.g., Singh Y N, Singh N N. CNS Drugs. 2002; 16 (11): 731-43.

In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 300 mg of Withania somnifera. In certain aspects, the compositions of the present disclosure comprise 300 mg of Withania somnifera. In certain aspects, the compositions of the present disclosure comprise about 400 mg of Withania somnifera. In certain aspects, the compositions of the present disclosure comprise 400 mg of Withania somnifera. In certain aspects, the compositions of the present disclosure comprise about 500 mg of Withania somnifera. In certain aspects, the compositions of the present disclosure comprise 500 mg of Withania somnifera. In certain aspects, the compositions of the present disclosure comprise about 600 mg of Withania somnifera. In certain aspects, the compositions of the present disclosure comprise 600 mg of Withania somnifera. In certain aspects, the compositions of the present disclosure comprise about 700 mg of Withania somnifera. In certain aspects, the compositions of the present disclosure comprise 700 mg of Withania somnifera.

In some aspects of the disclosure, the composition comprises an effective amount of Withania somnifera, and Piper methysticum. In some aspects, the compositions of the present disclosure comprise about 200 mg to about 700 mg of Withania somnifera, and about 25 mg to about 100 mg of Piper methysticum. In particular aspects, the compositions comprise 600 mg of Withania somnifera, and 52 mg of Piper methysticum.

In some aspects of the disclosure, the compositions comprise an effective amount of Withania somnifera, Griffonia simplicifolia, and Corydalis yanhusuo. In some aspects, the compositions comprise about 200 mg to about 700 mg of Withania somnifera, about 5 mg to about 100 mg of Griffonia simplicifolia, and about 100 mg to about 300 mg of Corydalis yanhusuo.

In certain aspects, the compositions of the present disclosure comprise Griffonia simplicifolia in the amount of about 5 mg, about 10 mg, about 15 mg, about 20 mg, about 25 mg, about 30 mg, about 35 mg, about 40 mg, about 45 mg, about 50 mg, about 55 mg, 60 mg, about 65 mg, about 70 mg, about 75 mg, about 80 mg, about 85 mg, about 90 mg, about 95 mg, or about 100 mg. In certain aspects, the compositions of the present disclosure comprise about 20 mg of Griffonia simplicifolia. In certain aspects, the compositions of the present disclosure comprise 20 mg of Griffonia simplicifolia. In certain aspects, the compositions of the present disclosure comprise about 40 mg of Griffonia simplicifolia. In certain aspects, the compositions of the present disclosure comprise 40 mg of Griffonia simplicifolia. In certain aspects, the compositions of the present disclosure comprise about 80 mg of Griffonia simplicifolia. In certain aspects, the compositions of the present disclosure comprise 80 mg of Griffonia simplicifolia. In certain aspects, the compositions of the present disclosure comprise about 100 mg of Griffonia simplicifolia. In certain aspects, the compositions of the present disclosure comprise 100 mg of Griffonia simplicifolia.

Griffonia is a genus of flowering plants in the legume family, Fabaceae. It belongs to the subfamily Cercidoideae. Griffonia species of the present disclosure include, but are not limited to Griffonia physocarpa, Griffonia speciose, Griffonia tessmannii and Griffonia simplicifolia. See, e.g., Baillon, H E. Adansonia 6:188. 1865.

Griffonia simplicifolia seeds produce a high concentration of 5-hydroxytryptophan (5-HTP). 5-HTP is an important building block for the human body to form serotonin, a neurotransmitter and thus Griffonia simplicifolia increases the concentration of serotonin. See, e.g., Lemaire P A, Adosraku R K. Phytochem Anal. 2002 November-December; 13 (6): 333-7.

In certain aspects, the compositions of the present disclosure comprise Corydalis yanhusuo in the amount of about 100 mg, about 110 mg, about 120 mg, 130 mg, about 140 mg, about 150 mg, about 160 mg, about 170 mg, about 180 mg, about 190 mg, 200 mg, about 210 mg, about 220 mg, 230 mg, about 240 mg, about 250 mg, about 260 mg, about 270 mg, about 280 mg, about 290 mg, or about 300 mg. In certain aspects, the compositions of the present disclosure comprise about 100 mg of Corydalis yanhusuo. In certain aspects, the compositions of the present disclosure comprise 100 mg of Corydalis yanhusuo. In certain aspects, the compositions of the present disclosure comprise about 150 mg of Corydalis yanhusuo. In certain aspects, the compositions of the present disclosure comprise 150 mg of Corydalis yanhusuo. In certain aspects, the compositions of the present disclosure comprise about 200 mg of Corydalis yanhusuo. In certain aspects, the compositions of the present disclosure comprise 200 mg of Corydalis yanhusuo. In certain aspects, the compositions of the present disclosure comprise about 300 mg of Corydalis yanhusuo. In certain aspects, the compositions of the present disclosure comprise 300 mg of Corydalis yanhusuo.

Corydalis is a genus of about 470 species of annual and perennial herbaceous plants in the family Papaveraceae. Corydalis species of the present disclosure include, but are not limited to Corydalis yanhusuo, Corydalis decumbens (e.g., Cheng C, et al., Biomed Res Int. 2019; 2019:9614781 (2019)), Corydalis mucronifera (e.g., Zhang J, et al., Phytochemistry, 159:199-207 (2019)), Corydalis bungeana (e.g., Yang C et al., Molecules., 21 (8): 975 (2016)), Corydalis tomentella (e.g., Wang Y M et al., Ming W Z, Bioorg Chem., 95:103489 (2020)), Corydalis saxicola (e.g., Liu X W et al., J Pharm Biomed Res Int., Biomed Anal. 159:252-261 (2018)), Corydalis ternata (e.g., Kim S R et al., Planta Med., 65 (3): 218-221 (1999), Corydalis turtschaninovii (e.g., Lee H et al., Int J Mol Sci., 18 (12): 2748 (2017)), Corydalis govaniana (e.g., Sivakumaran N et al., 2018:3171348 (2018)), Corydalis racemosa (e.g., Yao H N et al., Nat Prod Res. 1-7 (2019)), Corydalis edulis (e.g., Liang S. et al., J Ethnopharmacol., 251:112540 (2020)), Corydalis ambigua var. amurensis (e.g., Zhu X Z. et al., 86 Suppl 2:173-175 (1991)), Corydalis hendersonii (e.g., Bai R et al., J Ethnopharmacol., 207:174-183 (2017)), Corydalis calliantha (e.g., Wangchuk P et al., Phytother Res., 24 (4): 481-485 (2010)), Corydalis heterocarpa (e.g., Kang K H et al., Food Chem Toxicol., 47 (8): 2129-2134 (2009)), Corydalis ochotensis (e.g., Yu J J et al., 37 (10): 1795-1798 (2014)), Corydalis lutea (e.g., Boegge S C et al., Planta Med., 62 (2): 173-174 (1996)), and Corydalis dubia (e.g., Shepherd C et al., J Ethnopharmacol., 211:17-28 (2018)).

In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 300 mg of Withania somnifera. In certain aspects, the compositions of the present disclosure comprise 300 mg of Withania somnifera. In certain aspects, the compositions of the present disclosure comprise about 400 mg of Withania somnifera. In certain aspects, the compositions of the present disclosure comprise 400 mg of Withania somnifera. In certain aspects, the compositions of the present disclosure comprise about 500 mg of Withania somnifera. In certain aspects, the compositions of the present disclosure comprise 500 mg of Withania somnifera. In certain aspects, the compositions of the present disclosure comprise about 600 mg of Withania somnifera. In certain aspects, the compositions of the present disclosure comprise 600 mg of Withania somnifera. In certain aspects, the compositions of the present disclosure comprise about 700 mg of Withania somnifera. In certain aspects, the compositions of the present disclosure comprise 700 mg of Withania somnifera.

In some aspects of the disclosure, the compositions comprise an effective amount of Withania somnifera, Griffonia simplicifolia, and Corydalis yanhusuo. In some aspects, the compositions comprise about 200 mg to about 700 mg of Withania somnifera, about 5 mg to about 100 mg of Griffonia simplicifolia, and about 100 mg to about 300 mg of Corydalis yanhusuo. In particular aspects, the composition comprises 600 mg of Withania somnifera, 40 mg of Griffonia simplicifolia, and 200 mg of Corydalis yanhusuo.

In some aspects, the compositions further comprise hemp oil derived from a Cannabis plant. The oils can be derived from different parts of the plant including but not limited to hemp stalk, hemp leaf, hemp flower, and hemp seed. In certain aspects, the compositions of the present disclosure comprise hemp oil in the amount of about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg. In certain aspects, the compositions of the present disclosure comprise about 1 mg of hemp oil. In certain aspects, the compositions of the present disclosure comprise 1 mg of hemp oil. In certain aspects, the compositions of the present disclosure comprise about 2 mg of hemp oil. In certain aspects, the compositions of the present disclosure comprise 2 mg of hemp oil. In certain aspects, the compositions of the present disclosure comprise about 5 mg of hemp oil. In certain aspects, the compositions of the present disclosure comprise 5 mg of hemp oil. In certain aspects, the compositions of the present disclosure comprise about 10 mg of hemp oil. In certain aspects, the compositions of the present disclosure comprise 10 mg of hemp oil.

In some aspects, the compositions according to the present disclosure further comprise one or more cannabinoids. Cannabinoids are compounds found in Cannabis plants that target various aspects of the endocannabinoid system. Notable cannabinoids include cannabidiol (CBD), cannabinol (CBN), and tetrahydrocannabinol (THC)—the compound that produces the euphoric “high” from cannabis consumption. Cannabinoid consumption can have positive effects on, e.g., sleep, anxiety, mood, creativity, focus, and energy levels.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the compositions according to the present disclosure further comprise one or more flavorants. Flavorants are natural or artificial compounds that impart a pleasant flavor and odor to oral formulations. Exemplary flavorants include artificial, natural, and synthetic flavors such as vanilla, chocolate, pumpkin pie, cookie dough, “baked pear” spices, and citrus oils including lemon, orange, lime, grapefruit, yuzu, sudachi, and fruit essences including apple, green apple, pear, peach, grape, berry, blueberry, strawberry, wild berry, date, passion fruit, plum, pineapple, apricot, banana, melon, apricot, cherry, raspberry, blackberry, tropical fruit, lychee, mango, mangosteen, pomegranate, and papaya.

III. Methods of Increasing Focus, Stamina, Motivation, Sociability, and Recovery from Stress

The present disclosure further provides methods of increasing mental focus, increasing energy, and reducing stress in a subject. In some aspects, the disclosure provides methods of increasing mental focus in a subject, comprising administering to the subject one or more compositions of the present disclosure. In some aspects, the present disclosure provides methods of increasing mental energy and physiological energy in a subject, comprising administering to the subject one or more compositions of the present disclosure. In some aspects, the present disclosure provides methods of reducing mental stress in a subject, comprising administering to the subject one or more compositions of the present disclosure.

In some aspects, the methods of the present disclosure further comprise administering to the subject one or more cannabinoids. As used herein, a “cannabinoid” is a chemical compound (such as cannabinol, THC or cannabidiol) that is found in the plant species Cannabis. Cannabinoids can have positive effects on, e.g., sleep, anxiety, mood, creativity, focus, and energy levels.

### Methods of Increasing Mental Focus

The present disclosure provides methods of increasing mental focus in a subject, comprising administering to the subject one or more compositions of the present disclosure.

As described herein, the term “mental focus” or “concentration” refers to the ability of an individual to direct mental effort on the most relevant information in the environment allowing an individual the ability to stay focused on the pertinent cues of their environment. Mental focus is critical for learning new things, achieving goals, and performing well across a wide variety of situations.

A wide variety of tests measures mental focus, including a sustained attention test (SART) (See, e.g., S. M Silverstein, et al., Computers in Human Behavior, Volume 14, Issue 3, 1998, Pages 463-475; Smilek D, et al., Neuropsychologia. 2010 July; 48 (9): 2564-70.), attention-related cognitive errors (ARCES) (See, e.g., Cheyne J A, et al., Conscious Cogn. 2006 September; 15 (3): 578-92), Attentional Capacity Test (ACT), Continuous Performance Tests (CPTs), and Tests of Variable Attention (TOVA).

SART is a vigilance task designed to assess the ability of individuals to sustain attention to visual stimuli. ARCES is a self-report scale developed to evaluate everyday performance failures arising directly or primarily from brief failures of sustained attention. ACT, CPT, and TOVA are tests of selective, sustained, and divided attention, respectively.

The compositions of the present disclosure use an overlapping pharmacological pleiotropy to exploit the untapped synergistic activity of adaptogens. The compositions of the present disclosure herein target key receptors in the cholinergic, dopaminergic, serotonergic, adrenergic, GABAergic, and endocannabinoid systems. These systems are involved in cognition and executive functioning, thus the present compositions exploit these natural receptors thereby boosting mental focus and attention.

The administration of the compositions according to the present disclosure to a subject improve mental focus through the network pharmacology by reducing oxidative stress, stimulating the cholinergic system, activating the GABA-A receptors, increasing the concentration of monoamine neurotransmitters, and reducing inflammation.

The present disclosure provides methods for increasing mental focus in a subject, comprising administering to a subject one or more compositions described herein, wherein the one or more compositions reduces oxidative stress, stimulates the cholinergic system, activates the GABA-A receptors, increases the concentration of monoamine neurotransmitters, reduces inflammation, or any combination thereof. In some aspects, the methods of the present disclosure increase mental focus by reducing oxidative stress. In some aspects, the methods of the present disclosure increase mental focus by stimulating the cholinergic system. In some aspects, the methods of the present disclosure increase mental focus by activating the GABA-A receptors. In some aspects, the methods of the present disclosure increase mental focus by increasing the concentration of monoamine neurotransmitters. In some aspects, the methods of the present disclosure increase mental focus by reducing inflammations.

In some aspects, the present disclosure provides methods for increasing mental focus in a subject, comprising administering to a subject one or more compositions described herein, wherein the one or more compositions increases concentration in the subject. In some aspects, the present disclosure provides methods for increasing mental focus in a subject, comprising administering to a subject one or more compositions described herein, wherein the one or more compositions increases alertness in the subject.

In some aspects, the present disclosure provides methods for increasing mental focus in a subject, comprising administering to a subject one or more compositions described herein, wherein the one or more compositions increases concentration and alertness in the subject.

In some aspects, the methods of increasing mental focus in a subject comprise further administering to the subject one or more cannabinoids.

Cannabinoids are compounds found in Cannabis plants that target various aspects of the endocannabinoid system (ECS). The ECS has been implicated in a wide variety of physiological and pathophysiological processes, including neural development, immune function, inflammation, appetite, metabolism and energy homeostasis, cardiovascular function, digestion, bone development and bone density, synaptic plasticity and learning, pain, reproduction, psychiatric disease, psychomotor behavior, memory, wake/sleep cycles, and the regulation of stress and emotional state.

Cannabis consumption results in a wide range of benefits, from medicinal to recreational, however, reversible deficits in cognition have been observed in some subjects. Moreover, a survey demonstrated that over half of cannabis users perceived it to have negative effects on memory and focus. See, e.g., Brenton J N, et al. J Neurol. February 2018; 265 (2): 417-423. In some subjects, THC-comprising cannabinoids can disrupt neural signaling when binding to the receptors responsible for memory, resulting in loss of interest and problems with concentration.

The adaptogen formulations of the present disclosure are designed to offset these adverse effects through the synergistic activity between the adaptogen formulations and cannabis. The result is a more focused, “smart botanical” agent that provides the subject with a more controlled cannabis experience, guided by the adaptogen formulation of choice. Thus, in some aspects, the compositions of the present disclosure work synergistically with a cannabis product to provide a more controlled cannabis experience in a subject, guided by the formulation of the adaptogen composition. In a particular aspect, the compositions of the disclosure for increasing mental focus work synergistically with a cannabis product to provide the subject with more controlled feelings of focus, concentration and alertness.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the cannabinoids comprise CBD, CBN, THC, or any combination thereof. In some aspects, the cannabinoids comprise CBD and CBN. In some aspects, the cannabinoid comprises THC.

As used herein, combined administration (co-administration) includes simultaneous administration of the compositions in the same or different dosage form or separate administration of the compositions (e.g., sequential administration). In some aspects, the one or more compositions of the present disclosure are simultaneously administered with one or more cannabinoids. In some aspects, the one or more compositions of the present disclosure are sequentially administered with the administration of one or more cannabinoids. In some aspects, the one or more compositions of the present disclosure are administered after the administration of one or more cannabinoids. In some aspects, the one or more compositions of the present disclosure are administered before the administration of one or more cannabinoids.

In certain aspects, the one or more compositions of the disclosure are administered at predetermined time intervals over an extended period. In certain aspects, the one or more compositions are administered once a day. In certain aspects, the one or more compositions are administered once every day. In certain aspects, the one or more compositions are administered every other day. In certain aspects, the one or more compositions are administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In certain aspects, the compositions of the present disclosure are simultaneously administered (e.g., focus formulation #2 is administered with energy formulation #4). In certain aspects, the compositions of the present disclosure are sequentially administered (e.g., stress-relief formulation #5 can be administered first followed by (e.g., immediately followed by) the administration of focus formulation #1), or any combination thereof.

### Methods for Increasing Motivation, Stamina and Sociability

The present disclosure further provides methods for increasing mental energy and physiological energy in a subject, comprising administering to the subject one or more compositions of the present disclosure. The present disclosure further provides methods for increasing motivation and stamina in a subject, comprising administering to the subject one or more compositions of the present disclosure.

As described herein, “mental energy” refers to the ability to participate in cognitive work. Mental energy is a mood state, comprising feelings of productivity, motivation to do mental work, and feelings of energy and/or absence of feelings of fatigue. “Physiological energy” refers to the body's the capacity to perform work and it is a result of increased mental energy, reflected in, e.g., a change in blood pressure and an increased ability to perform a physical task. “Stamina” refers to the ability of an organism to exert itself and remain active for a period of time. “Motivation” according to the present invention refers to a feeling of actively and positively tackling the above activities, and “increasing motivation” refers to enhancing the feeling. As used herein, the term “improving sociability” refers to increasing the willingness to talk and engage in activities with other people.

Individuals who desire an energy boost often consume coffee or other caffeine-containing energy beverages, along with high-sugar and high-fat snacks. If consumed at the correct dose, caffeine can provide immediate effects to relieve tiredness. However, overconsumption of caffeine-containing products can cause sleeplessness and hypertension. The compositions of the present invention can provide the energy boost necessary while minimizing the adverse effects of high concentrations of caffeine through synergistic effects on the stress-response system.

The compositions of the present disclosure target energy metabolism globally through the regulation of hormones such as corticosteroids and insulin. Thus, the disclosed compositions exploit these natural biological processes involved in energy production and metabolism, thereby boosting mental and physical energy. Energy production and storage in the body are dependent on phosphorylated compounds, such as adenosine triphosphate (ATP) and creatine phosphate. The compositions of the present disclosure work naturally to promote the synthesis of ATP, the primary energy source for all cells in the body. Thus, the present disclosure provides methods for increasing mental energy and physiological energy in a subject, comprising administering to the subject one or more compositions disclosed herein, wherein the method promotes the synthesis of ATP in the subject.

The compositions of the present disclosure use an overlapping pharmacological pleiotropy to exploit the synergistic activity of adaptogens. The combinations of adaptogens in the compositions herein improve energy metabolism resulting in increased mental energy and physiological energy. By improving overall health and restoring balance, the botanical blends of adaptogens of the present disclosure provide more mental energy and physiological energy and greater well-being.

In some aspects, the present disclosure provides methods for increasing mental energy in a subject, comprising administering to the subject one or more compositions of the disclosure. In some aspects, the present disclosure provides methods for increasing physiological energy in a subject, comprising administering to the subject one or more compositions of the disclosure. In some aspects, the present disclosure provides methods for increasing mental energy and physiological energy in a subject, comprising administering to the subject one or more compositions of the disclosure.

The administration of the compositions according to the present disclosure to a subject increase mental energy and physiological energy through the network pharmacology by blocking the effects of adenosine, reducing oxidative stress, stimulating the cholinergic system, increasing the concentration of monoamine neurotransmitters, and reducing inflammation.

The present disclosure provides methods for increasing mental energy and physiological energy in a subject, comprising administering to a subject one or more compositions described herein, wherein the one or more compositions blocking the effects of adenosine, reduces oxidative stress, stimulates the cholinergic system, increases the concentration of monoamine neurotransmitters, reduces inflammation, or any combination thereof. In some aspects, the methods of the present disclosure increase mental energy and physiological energy by blocking the effects of adenosine. In some aspects, the methods of the present disclosure increase mental energy and physiological energy by reducing oxidative stress. In some aspects, the methods of the present disclosure increase mental energy and physiological energy by stimulating the cholinergic system. In some aspects, the methods of the present disclosure increase mental energy and physiological energy by increasing the concentration of monoamine neurotransmitters. In some aspects, the methods of the present disclosure increase mental energy and physiological energy by reducing inflammation.

In some aspects, administering the compositions according to the present disclosure to a subject results in a noticeable energy boost. In some aspects, administering the compositions according to the present disclosure to a subject results in increased physical activity. In some aspects, administering the compositions according to the present disclosure to a subject results in increased mental activity.

In some aspects, the methods for increasing mental energy and physiological energy in a subject comprise further administering to the subject one or more cannabinoids.

Cannabinoids are compounds found in Cannabis plants that target various aspects of the endocannabinoid system (ECS). The ECS has been implicated in a wide variety of physiological and pathophysiological processes, including neural development, immune function, inflammation, appetite, metabolism and energy homeostasis, cardiovascular function, digestion, bone development and bone density, synaptic plasticity and learning, pain, reproduction, psychiatric disease, psychomotor behavior, memory, wake/sleep cycles, and the regulation of stress and emotional state.

Cannabis consumption results in a wide range of benefits, from medicinal to recreational, however in some subjects it has been known to increase fatigue, and decrease arousal, vigor, and elation. These subjects do not experience strain-specific effects. The compositions of the present disclosure were formulated to offset this adverse effect through the synergistic activity between the composition's adaptogens and cannabis. The compositions of the present disclosure were formulated to increase endurance and decrease fatigue states in subjects.

The adaptogen formulations of the present disclosure are designed to offset these adverse effects through the synergistic activity between the adaptogen formulations and cannabis. The result is a more focused, “smart botanical” agent that provides the subject with a more controlled cannabis experience, guided by the adaptogen formulation of choice. Thus, in some aspects, the compositions of the present disclosure work synergistically with a cannabis product to provide a more controlled cannabis experience in a subject, guided by the formulation of the adaptogen composition. In a particular aspect, the compositions of the disclosure for increasing mental energy and physiological energy work synergistically with a cannabis product to provide the subjects more controlled energy boost and endurance.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the cannabinoids comprise CBD, CBN, THC, or any combination thereof. In some aspects, the cannabinoids comprise CBD and CBN. In some aspects, the cannabinoid comprises THC.

As used herein, combined administration (co-administration) includes simultaneous administration of the compositions in the same or different dosage form or separate administration of the compositions (e.g., sequential administration). In some aspects, the one or more compositions of the present disclosure are simultaneously administered with one or more cannabinoids. In some aspects, the one or more compositions of the present disclosure are sequentially administered with the administration of one or more cannabinoids. In some aspects, the one or more compositions of the present disclosure are administered after the administration of one or more cannabinoids. In some aspects, the one or more compositions of the present disclosure are administered before the administration of one or more cannabinoids.

In some aspects, the methods for increasing mental energy and physiological energy in a subject comprise administering to the subject one or more compositions of the disclosure in combination with one or more cannabinoids, wherein the effects of the cannabinoid in the subject are heightened, providing a more meaningful and deeper mind and body experience. In some aspects, the effects of the cannabinoid in the subject comprise changes in mental focus, mood, calmness, sleep quality, productivity, creativity, or any combination thereof.

In certain aspects, the one or more compositions of the disclosure are administered at predetermined time intervals over an extended period. In certain aspects, the one or more compositions are administered once a day. In certain aspects, the one or more compositions are administered once every day. In certain aspects, the one or more compositions are administered every other day. In certain aspects, the one or more compositions are administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In certain aspects, the compositions of the present disclosure are simultaneously administered (e.g., focus formulation #2 is administered with energy formulation #4). In certain aspects, the compositions of the present disclosure are sequentially administered (e.g., stress-relief formulation #5 can be administered first followed by (e.g., immediately followed by) the administration of focus formulation #1), or any combination thereof.

Methods for Recovery from Environmental, Physical, and Mental Stress

The present disclosure further provides methods for reducing mental stress in a subject, comprising administering to the subject one or more compositions of the present disclosure. The present disclosure further provides methods for recovering from stress in a subject, comprising administering to the subject one or more compositions of the present disclosure.

Environmental, physical and mental stress is the body's response to external and internal pressures, and can be caused by many different stimuli. For example, mental stress can be induced by new experiences, unexpected experiences, threatening experiences, or when feeling a loss of control. Mental stress includes, e.g., feeling anxious, afraid, angry or aggressive, sad, irritable, frustrated, depressed, or any combination thereof. Mental stress may also include feelings of being overwhelmed or unable to cope with mental or emotional pressure. Each person deals with mental stress differently, depending on genetics, life experiences, personality, and social and economic circumstances.

The administration of the compositions according to the present disclosure to a subject reduce mental stress through the network pharmacology by blocking the effects of adenosine, reducing oxidative stress, stimulating the cholinergic system, increasing the concentration of monoamine neurotransmitters, reducing inflammation, and upregulating levels of plasma β-endorphin and hypothalamic proopiomelanocortin.

The present disclosure provides methods for reducing mental stress in a subject, comprising administering to a subject one or more compositions described herein, wherein the one or more compositions block the effects of adenosine, reduce oxidative stress, stimulate the cholinergic system, increase the concentration of monoamine neurotransmitters, reduce inflammation, upregulate levels of plasma β-endorphin and hypothalamic proopiomelanocortin, or any combination thereof. In some aspects, the methods of the present disclosure reduce mental stress by blocking the effects of adenosine. In some aspects, the methods of the present disclosure reduce mental stress by reducing oxidative stress. In some aspects, the methods of the present disclosure reduce mental stress by stimulating the cholinergic system. In some aspects, the methods of the present disclosure reduce mental stress by increasing the concentration of monoamine neurotransmitters. In some aspects, the methods of the present disclosure reduce mental stress by reducing inflammation. In some aspects, the methods of the present disclosure reduce mental stress by upregulating levels of plasma β-endorphin and hypothalamic proopiomelanocortin.

In certain aspects, the disclosure provides methods for reducing mental stress in a subject, comprising administering to a subject one or more compositions described herein, wherein the subject's mood is elevated after administration of the one or more compositions. In certain aspects, the disclosure provides methods for reducing mental stress in a subject, comprising administering to a subject one or more compositions described herein, wherein the subject's ability to fall asleep is improved after administration of the one or more compositions. In certain aspects, the disclosure provides methods for reducing mental stress in a subject, comprising administering to a subject one or more compositions described herein, wherein the subject's quality of sleep is improved after administration of the one or more compositions. In some aspects, the subject has a sleep disorder. In some aspects, the subject has insomnia.

In certain aspects, the disclosure provides methods for reducing mental stress in a subject, comprising administering to a subject one or more compositions described herein, wherein the mental stress is associated with a sleep disorder, a panic disorder, a social anxiety disorder, a generalized anxiety disorder, a post-traumatic stress disorder, a depressive disorder, or any combination thereof.

In some aspects, the methods for reducing mental stress in a subject comprise further administering to the subject one or more cannabinoids.

Cannabinoids are compounds found in Cannabis plants that target various aspects of the endocannabinoid system (ECS). The ECS has been implicated in a wide variety of physiological and pathophysiological processes, including neural development, immune function, inflammation, appetite, metabolism and energy homeostasis, cardiovascular function, digestion, bone development and bone density, synaptic plasticity and learning, pain, reproduction, psychiatric disease, psychomotor behavior, memory, wake/sleep cycles, and the regulation of stress and emotional state.

Cannabis consumption results in a wide range of benefits, from medicinal to recreational, however, it has been known to increase depression and anxiety, and decreased positive mood, friendliness in some subjects. Moreover, these subjects do not experience the beneficial effects of cannabinoids on stress and pain. The compositions of the present disclosure were formulated to offset this adverse effect through the synergistic activity between the composition's adaptogens and cannabis resulting in decreased anxiety, improved mood, etc. In certain aspects, the compositions of the disclosure provide anti-inflammatory benefits which when combined with the analgesic properties of cannabis products results in a reduction of pain and physiological stress response.

The compositions of the present disclosure were formulated to reduce psychological and physiological stress in a subject.

The compositions of the present disclosure were formulated to enhance recovery from environmental, psychological and physiological stress in a subject.

The adaptogen formulations of the present disclosure are designed to offset these adverse effects through the synergistic activity between the adaptogen formulations and cannabis. The result is a more focused, “smart botanical” agent that provides the subject with a more controlled cannabis experience, guided by the adaptogen formulation of choice. Thus, in some aspects, the compositions of the present disclosure work synergistically with a cannabis product to provide a more controlled cannabis experience in a subject, guided by the formulation of the adaptogen composition. In a particular aspect, the compositions of the disclosure for reducing stress work synergistically with a cannabis product to provide the subjects more controlled mood enhancement, pain relief and reduction of psychological and physiological stress.

In some aspects, the one or more cannabinoids are selected from tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, and trihydroxy-delta-9-tetrahydrocannabinol.

In some aspects, the cannabinoids comprise CBD, CBN, THC, or any combination thereof. In some aspects, the cannabinoids comprise CBD and CBN. In some aspects, the cannabinoid comprises THC.

The botanical blends of adaptogens in the present compositions act as “smart botanical agents,” homing through the individual's body to go to the greatest area(s) of needs and causes. When combined with administration of one or more cannabinoids, the compositions of the present disclosure reduce mental stress in the subject while further enhancing the mind and body benefits of the one or more cannabinoids.

As used herein, combined administration (co-administration) includes simultaneous administration of the compositions in the same or different dosage form or separate administration of the compositions (e.g., sequential administration). In some aspects, the one or more compositions of the present disclosure are simultaneously administered with one or more cannabinoids. In some aspects, the one or more compositions of the present disclosure are sequentially administered with the administration of one or more cannabinoids. In some aspects, the one or more compositions of the present disclosure are administered after the administration of one or more cannabinoids. In some aspects, the one or more compositions of the present disclosure are administered before the administration of one or more cannabinoids.

In some aspects, the methods for reducing mental stress in a subject comprise administering to the subject one or more compositions of the disclosure in combination with one or more cannabinoids, wherein the effects of the cannabinoid in the subject are heightened, providing a more meaningful and deeper mind and body experience. In some aspects, the effects of the cannabinoid in the subject comprise changes in mental focus, mood, calmness, sleep quality, productivity, creativity, or any combination thereof.

In certain aspects, the one or more compositions of the disclosure are administered at predetermined time intervals over an extended period. In certain aspects, the one or more compositions are administered once a day. In certain aspects, the one or more compositions are administered once every day. In certain aspects, the one or more compositions are administered every other day. In certain aspects, the one or more compositions are administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In certain aspects, the compositions of the present disclosure are simultaneously administered (e.g., focus formulation #2 is administered with energy formulation #4). In certain aspects, the compositions of the present disclosure are sequentially administered (e.g., stress-relief formulation #5 can be administered first followed by (e.g., immediately followed by) the administration of focus formulation #1), or any combination thereof.

### IV. Methods for Alleviating Hypothalamic-Pituitary-Adrenal (HPA) Axis Dysfunction

The present disclosure further provides methods for alleviating hypothalamic-pituitary-adrenal (HPA) axis dysfunction in a subject, comprising administering to the subject one or more compositions of the present disclosure.

The endocannabinoid system (ECS) is characterized by two known cannabinoid receptors in the human body, CB1, which is primarily located in the central nervous system, and CB2, which is found mainly in the immune system and blood cells. The ECS has been implicated in a wide variety of physiological and pathophysiological processes, including neural development, immune function, inflammation, appetite, metabolism and energy homeostasis, cardiovascular function, digestion, bone development and bone density, synaptic plasticity and learning, pain, reproduction, psychiatric disease, psychomotor behavior, memory, wake/sleep cycles, and the regulation of stress and emotional state.

The endocannabinoid signaling system is one of the critical regulators of the stress response and is essential for a proper return to the non-stressed state. The endocannabinoid system constrains the magnitude of the stress response, promotes the recovery of the hypothalamus-pituitary-adrenal (HPA) axis to non-stressed levels, and facilitates habituation of the stress response to repeated or ongoing stress. It also directly inhibits stress-associated processes such as fear, anxiety, depressive behaviors, inflammation, and hyperalgesia and promotes behaviors inhibited by the stress response such as feeding and sleep.

These cannabinoid receptors are naturally present and activated by endocannabinoids produced by the human body for neural and cell signaling. In neurons, endocannabinoids bind to the CB1 receptors at the pre-synaptic junction and, among other effects, impact the release of GABA.

Consumption of cannabis products can exert a variety of beneficial effects by acting on the ECS, including effects on sleep, anxiety, mood, creativity, focus, and energy levels. Cannabis products comprising tetrahydrocannabinol are known to elicit a euphoric feeling or “high.” The cannabis high varies from individual to individual, depending on many factors, including the strain of Cannabis plant, the amount consumed, the method of consumption, the biochemistry of the individual consuming it, and the individual's level of experience in consuming cannabis.

In addition to recreational use, cannabis products have a broad scope of medical applications, including, but not limited to, treatment of epilepsy, multiple sclerosis spasms, anxiety disorders, bipolar disorder, nausea, convulsion, inflammation, as well as inhibiting cancer cell growth.

Signaling by the ECS is known to regulate the hypothalamic-pituitary-adrenocortical (HPA) axis in the context of a stress response. Chronic overstimulation of the ECS, such as through chronic consumption of cannabis products, can have negative effects on the HPA axis and homeostasis within the body. Chronic cannabis consumption can case, e.g., blunted adrenocorticotropic hormone (ACTH) and cortisol reactivity in response to acute stress, resulting in a dysregulated HPA axis. HPA axis dysfunction can manifest in numerous symptoms, including fatigue, disturbed sleep, anxiety, immunosuppression, adrenal suppression, hypocortisolism, adverse effect on brain function, including learning and memory, and changes in body weight

Provided herein are methods for alleviating HPA axis dysfunction in a subject, comprising administering to the subject one or more compositions disclosed herein. In some aspects, the HPA axis dysfunction in the subject is a result of the subject's cannabis consumption. In some aspects, the HPA axis dysfunction in the subject causes fatigue, anergia, disturbed sleep, anxiety, immunosuppression, adrenal suppression, hypocortisolism, adrenocorticotropic hormone (ACTH) suppression, adverse effect on brain function, including learning and memory, changes in body weight, or any combination thereof.

Administering one or more compositions of the present disclosure in combination with one or more cannabis products capitalizes on the benefits of cannabis products while alleviating adverse effects on the HPA axis, thereby allowing for a more controlled and directed experience. When combined with cannabis consumption, the compositions of the present disclosure provide the beneficial effects of alleviating HPA axis dysfunction with controlled and directed cannabis effects, resulting in a “healthy high.”

Thus, provided herein are methods of alleviating HPA axis dysfunction in a subject while concurrently providing a controlled and directed cannabis experience in the subject, comprising administering to the subject one or more compositions of the disclosure in combination with one or more cannabis products. In some aspects, the one or more compositions are administered to the subject before the administering of one or more cannabis products. In some aspects, the one or more compositions of the disclosure are administered to the subject after the administering the one or more cannabis products.

In certain aspects, the one or more compositions of the disclosure are administered at predetermined time intervals over an extended period. In certain aspects, the one or more compositions are administered once a day. In certain aspects, the one or more compositions are administered once every day. In certain aspects, the one or more compositions are administered every other day. In certain aspects, the one or more compositions are administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In certain aspects, the compositions of the present disclosure are simultaneously administered (e.g., focus formulation #2 is administered with energy formulation #4). In certain aspects, the compositions of the present disclosure are sequentially administered (e.g., stress-relief formulation #5 can be administered first followed by (e.g., immediately followed by) the administration of focus formulation #1), or any combination thereof.

### V. Formulations

The compositions of the present disclosure can be formulated for oral delivery. Oral forms of the compositions described herein can be solid or liquid. Suitable dosage forms can be candy, lip balm, tablets, capsules, pills, granules, suspensions, emulsions, syrups, or solutions.

In some aspects, the oral forms of administration are edible. Suitable dosage forms include, but are not limited to, lozenge, candy, wafer, tablet, film, spray, gummies, and gums.

In some aspects, the compositions disclosed herein are formulated as lozenges. In some aspects, the lozenges may include hard lozenges, soft lozenges, gummy lozenges, or chewable lozenges.

In some aspects, the compositions disclosed herein are formulated as candies. In some aspects, the candy may include hard candy, soft candy, gummy candy, or chewy candy.

In some aspects, the compositions disclosed herein are formulated as films, e.g., dissolvable films. The film, by way of non-limiting example, is a clear or opaque, flexible, thin material.

In some aspects, the compositions disclosed herein are formulated as gums. In some aspects, the gum may be non-degradable chewing gum, degradable chewing gum, or liquid filled chewing gum.

In some aspects, the compositions disclosed herein are formulated as lip balms. In some aspects, the lip balm may be in a tube, stick, or pot.

The following examples are offered by way of illustration and not by way of limitation.

### Examples

### Example 1: Adaptogen Compositions for Focus, Energy, and Stress

Exemplary focus formulations are described in Table 1.

TABLE 1

| | Ingredient | (mg) | Specifications |
|---|---|---|---|
| Focus Formulation | Salvia rosmarinus | 300 | 6% Carnosic acid |
| Focus - 1 | Panax quinquefolius | 200 | Cereboost |
| | Sceletium tortuosum | 100 | Zembrin |
| | Totals | 600 | |
| Focus Formulation | Panax quinquefolius | 150 | Cereboost |
| Focus - 2 | Citicoline | 250 | NLT 97% |
| | L-Theanine | 150 | NLT 97% |
| | Hemp oil | 2 | |
| | Totals | 552 | |

Exemplary energy boost formulations are described in Table 2.

TABLE 2

| | Ingredient | (mg) | Specifications |
|---|---|---|---|
| Energy Formulation | Rhodiola rosea | 250 | 5% rosavins 1.5% |
| Energy - 3 | | | salidrosides |
| | Paullinia cupana | 180 | 22% caffeine |
| | Sceletium tortuosum | 35 | Zembrin |
| | Hemp oil | 2 | |
| | Totals | 467 | |
| Energy Formulation | Withania somnifera | 320 | KSM-66 |
| Energy - 4 | Schisandra chinensis | 320 | 9% schisandrins |
| | Ilex paraguariensis | 160 | 15% caffeine |
| | Hemp oil | 2 | |
| | Totals | 802 | |

Exemplary stress relief formulations are described in Table 3.

TABLE 3

| | Ingredient | (mg) | Specifications |
|---|---|---|---|
| Stress Relief | Withania somnifera | 400 | KSM-66 |
| Formulation | Melissa officinalis L. EO | 0.5 | Essential oil |
| Stress - 5 | Melissa officinalis L | 100 | |
| | Passiflora incarnata | 200 | Concentrate |
| | Totals | 700.5 | |
| Stress Relief | Withania somnifera | 600 | KSM-66 |
| Formulation | Piper methysticum | 52 | 70% kavalactone |
| Stress - 6 | Totals | 600 | |
| Stress Relief | Withania somnifera | 560 | KSM-66 |
| Formulation | Corydalis yanhusuo | 200 | Draco |
| Stress - 7 | Griffonia simplicifolia | 40 | |
| | Hemp oil | 2 | |
| | Totals | 802 | |

### Example 2: Adaptogen Compositions for Focus, Motivation, Sociability, and Recovery from Stress

Exemplary focus formulations are described in Table 4.

TABLE 4

| | Ingredient | (mg) | Specifications |
|---|---|---|---|
| Focus Formulation | Acetyl-L-carnitine | 300 | NLT 98% |
| Focus - 5 | Citicoline | 250 | NLT 97% |
| | L-Theanine | 150 | NLT 97% |
| | Panax quinquefolius | 85 | NTL 4% |
| | | | ginsenosides |
| | Totals | 785 | |
| Focus Formulation | Acetyl-L-carnitine | 300 | NLT 98% |
| Focus - 5 | Citicoline | 250 | NLT 97% |
| | L-Theanine | 150 | NLT 97% |
| | Panex ginseng | 85 | NTL 4% |
| | | | ginsenosides |
| | Totals | 785 | |

Exemplary energy boost formulations are described in Table 5.

TABLE 5

| | Ingredient | (mg) | Specifications |
|---|---|---|---|
| Stamina and | Rhodiola rosea | 250 | NTL 5% rosavins; |
| Motivation | | | NTL 1.8% salidrosides |
| Formulation | Paullinia cupana | 180 | NLT 22% caffeine |
| | Sceletium tortuosum | 35 | NLT 0.2-0.5% |
| | | | alkaloids |
| | Totals | 465 | |
| Sociability | Withania somnifera | 318 | NLT 5% Withanolides |
| Formulation | Schisandra chinensis | 220 | NLT 9% schisandrins |
| | Ilex paraguariensis | 160 | NLT 15% caffeine; |
| | | | NLT 30% polyphenols |
| | Grifffonia simplicifolia | 100 | NLT 98% 5-HTP |
| | Totals | 798 | |

Exemplary stress recovery formulations are described in Table 6.

TABLE 6

| | Ingredient | (mg) | Specifications |
|---|---|---|---|
| Stress Recovery | Withania somnifera | 478 | NLT 5% withanolides |
| Formulation | Corydalis yanhusuo | 200 | NLT 50% THP |
| | Silybum marianum | 80 | NLT 80% silymarin |
| | Griffonia simplicifolia | 40 | NLT 98% 5-HTP |
| | Totals | 798 | |

### Example 3: Adaptogen Compositions for Controlled Cannabis Experience

The consumer will select the particular composition suited to their specific need (e.g., increased mental focus, increased mental energy and physiological energy, reduced stress). The consumer will consume the composition of choice along with their favorite cannabis product, resulting in a more controlled cannabis experience. For example, a subject experiencing mental stress from life events may wish to consume a cannabis product to relieve stress. However, the subject may be disinclined to consume a cannabis product to due to undesirable side effects such as increased depression, anxiety and paranoia. The subject will select a composition of the disclosure for reducing stress (e.g., Stress-7), and consume the composition in combination with a cannabis product. The subject will experience controlled and directed feelings of relaxation and stress relief without increased depression, anxiety and paranoia. Furthermore, the compositions will positively regulate the consumer's neuroendocrine system resulting in reduced corticosteroids and reduced physiological stress.

### Example 4: Adaptogen Compositions as a Cannabinoid Adjuvant

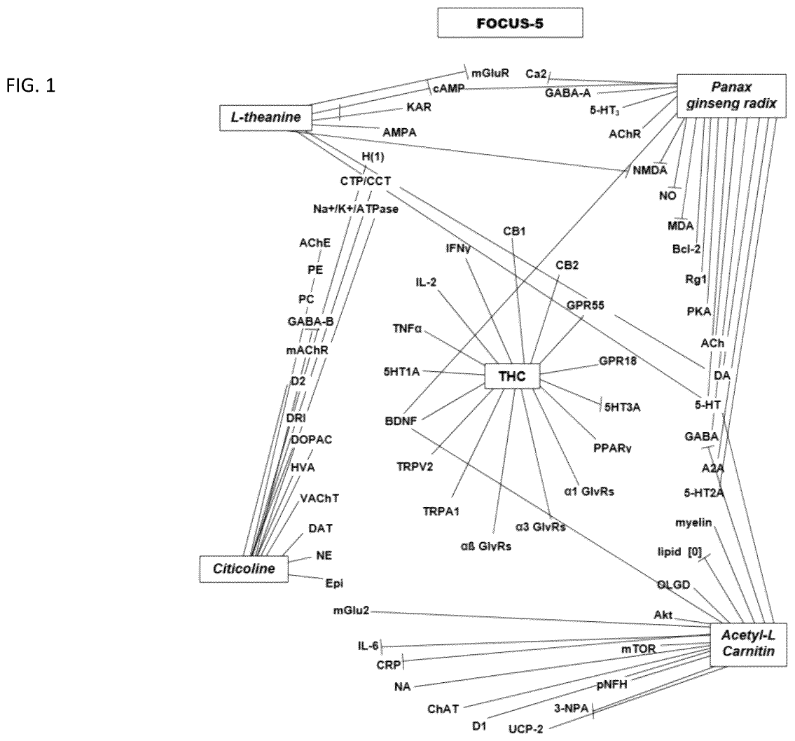

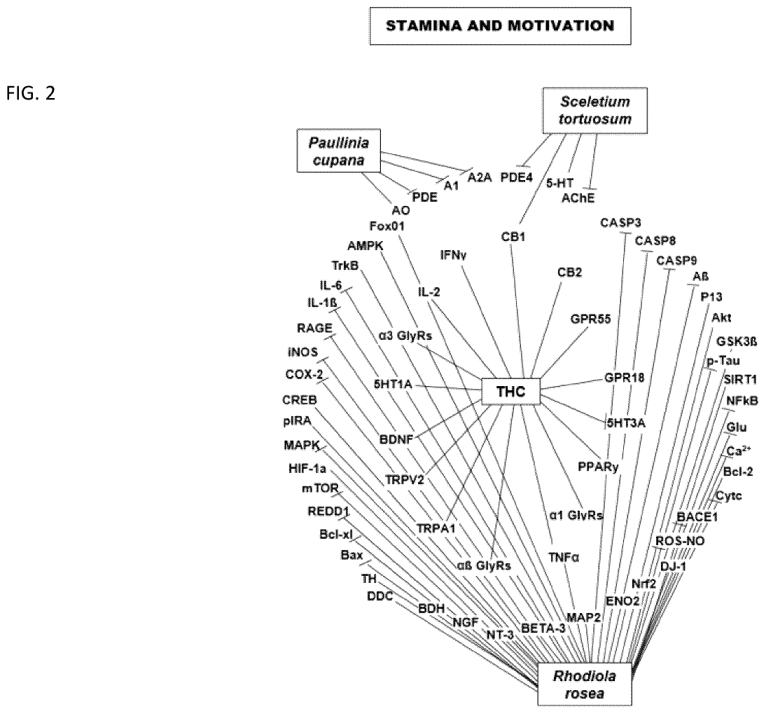

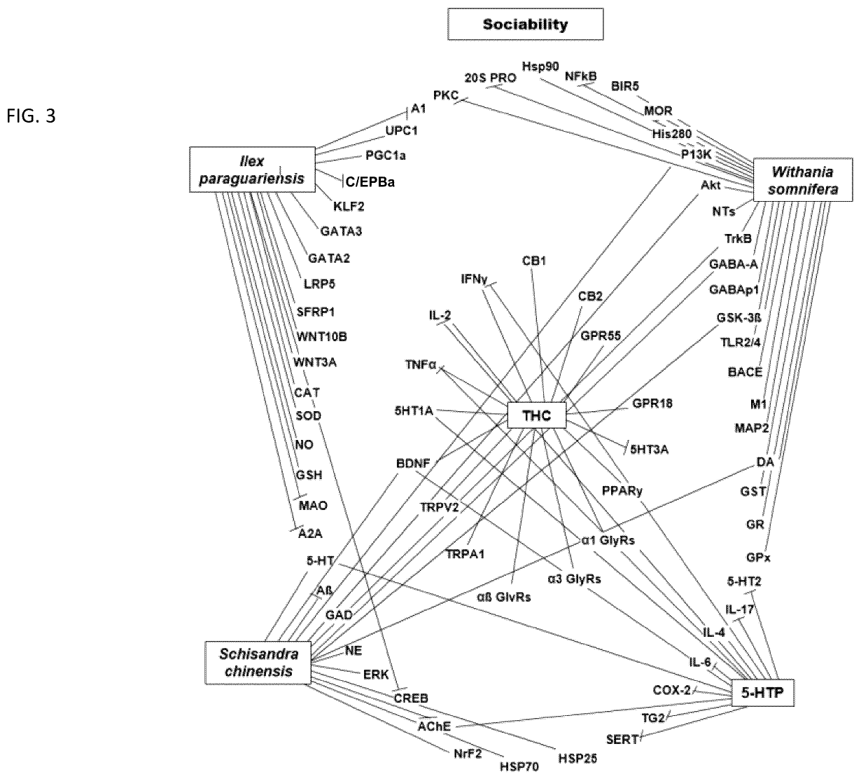

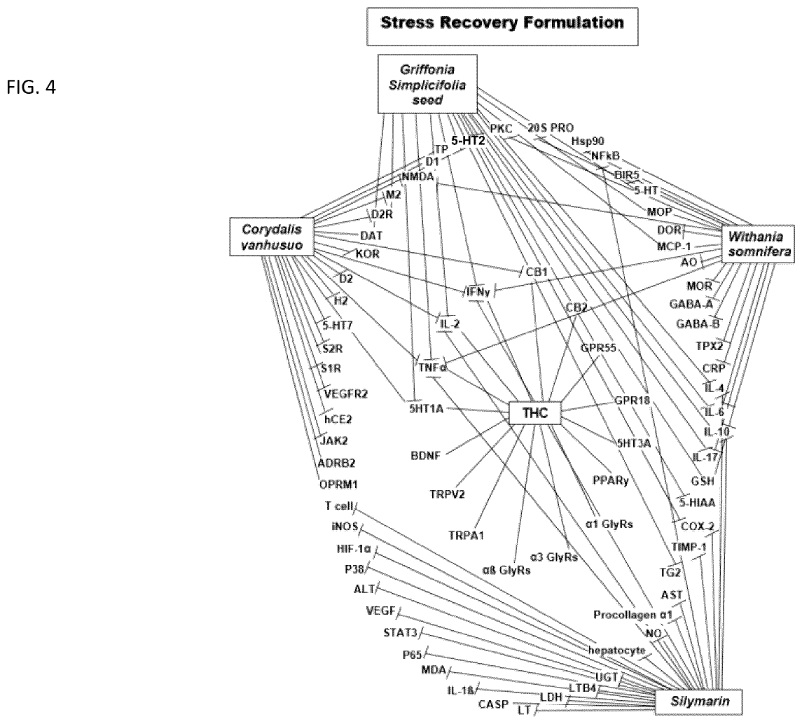

The formulations according to the present disclosure activate a host of pharmacological pathways by leveraging principles of network pharmacology. For example, FIG. 1 depicts the network pharmacology for a composition of the present disclosure related to increasing mental focus. Similarly FIG. 2 depicts the network pharmacology for a composition of the present disclosure related to increasing stamina and motivation. FIG. 3 depicts the network pharmacology for a composition of the present disclosure related to increasing sociability. FIG. 4 depicts the network pharmacology for a composition of the present disclosure related to stress recovery.

Leveraging this multi-faceted approach in the compositions of the present disclosure resulted in a variety of surprising and unexpected results. For example, the compositions of the present disclosure provided not only acute, but long-term beneficial effects on gastrointestinal (GI) stress response, mood, cognition, and fatigue. In this example, subjects selected 2 different compositions that best matched their particular needs and participated in a 10-day trial of combining the compositions with consumption of a cannabinoid product. The subjects kept track of their daily results, and provided feedback after the trial period.

82% of the subjects participating in the in-home trial indicated that the formulations according to the present disclosure positively impacted their overall cannabis experience. This included 29% of the subjects who experienced surprising and unpredictable long-term effects in the days following consumption of the formulations. These surprising results included reduction in GI stress response, reduction in feelings of isolation and reduction of fatigue. Additionally, 89% of the subjects reported an improvement in their ability to cope with various mental and physical stressors.

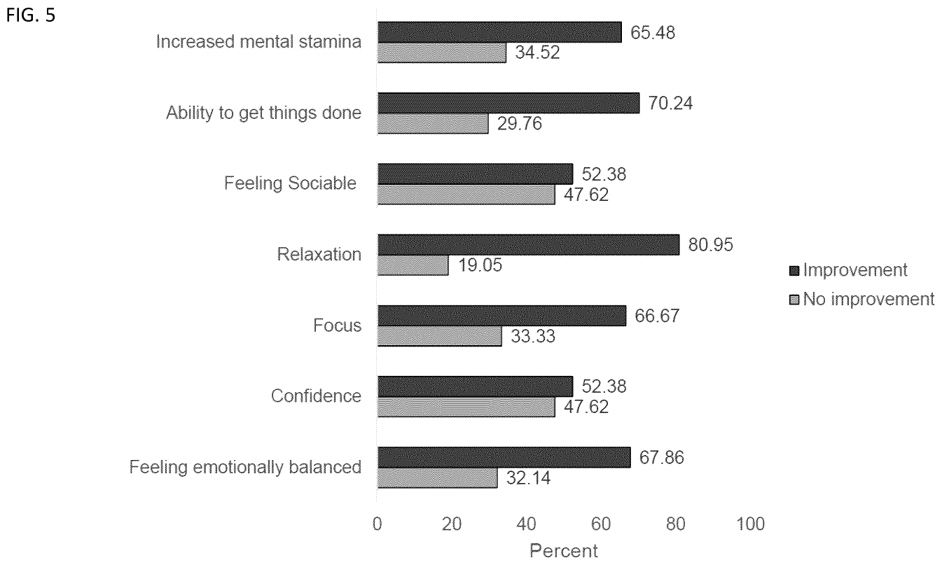

The compositions of the present disclosure also positively affected mental stressors when combined with a cannabis product. 65.48% of the subjects reported an increase in mental stamina; 67.86% of subjects reported feeling emotionally balanced; and 70.24% reported an improvement in their ability to get things done (FIG. 5).

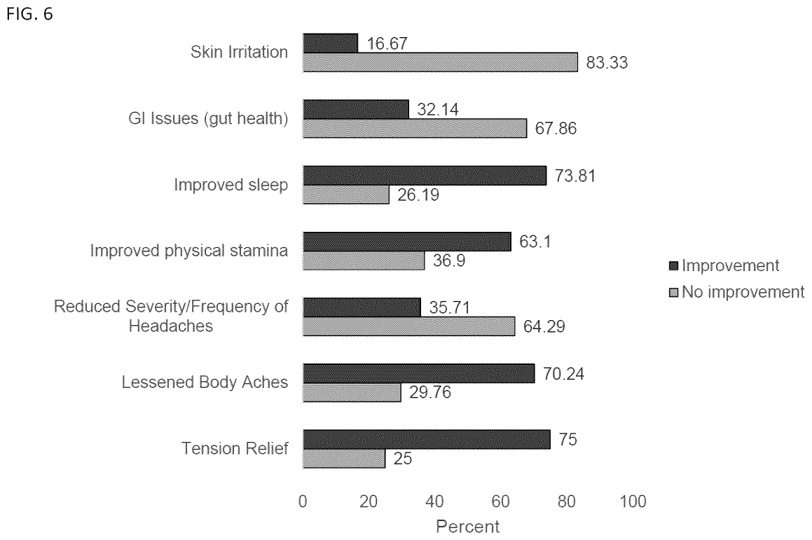

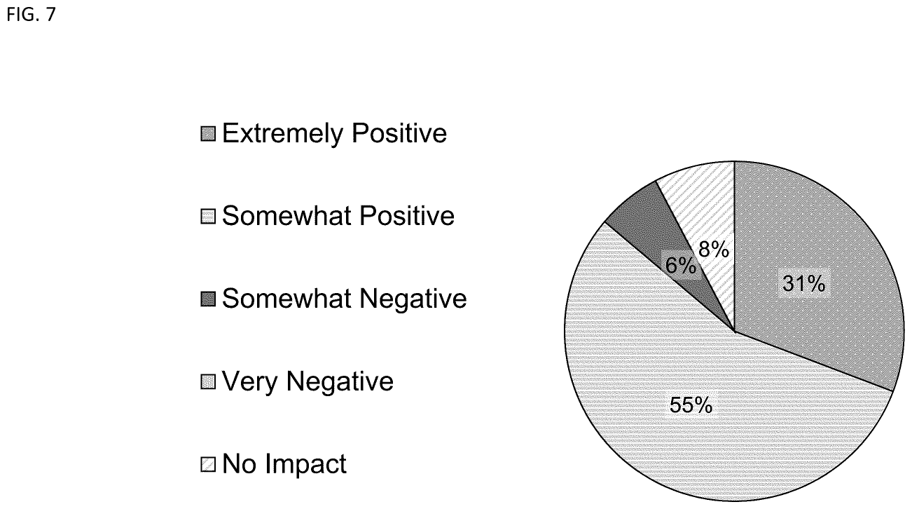

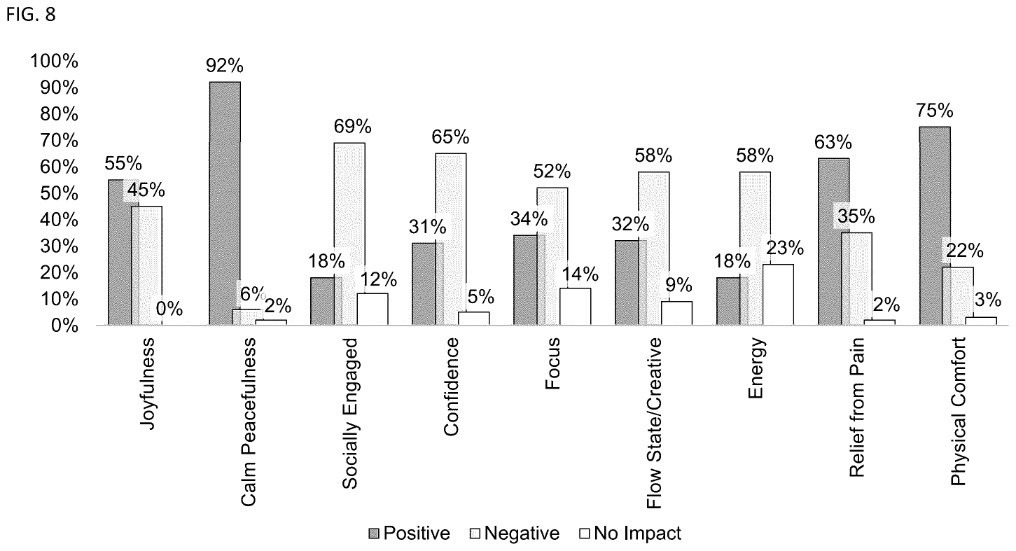

The compositions of the present disclosure also positively affected physical stressors when combined with a cannabis product. 75% of the subjects reported tension relief; 70.24% of the subjects reported reduced body aches; and 63% of the subjects reported improved physical stamina (FIG. 6). Additionally, approximately 86% of the subjects who used stress recovery formulation reported a positive experience when combining the composition with a cannabis product (FIG. 7). Furthermore, 75% of the same subjects reported physical comfort; 63% reported pain relief; and 92% reported the feeling of peacefulness (FIG. 8).

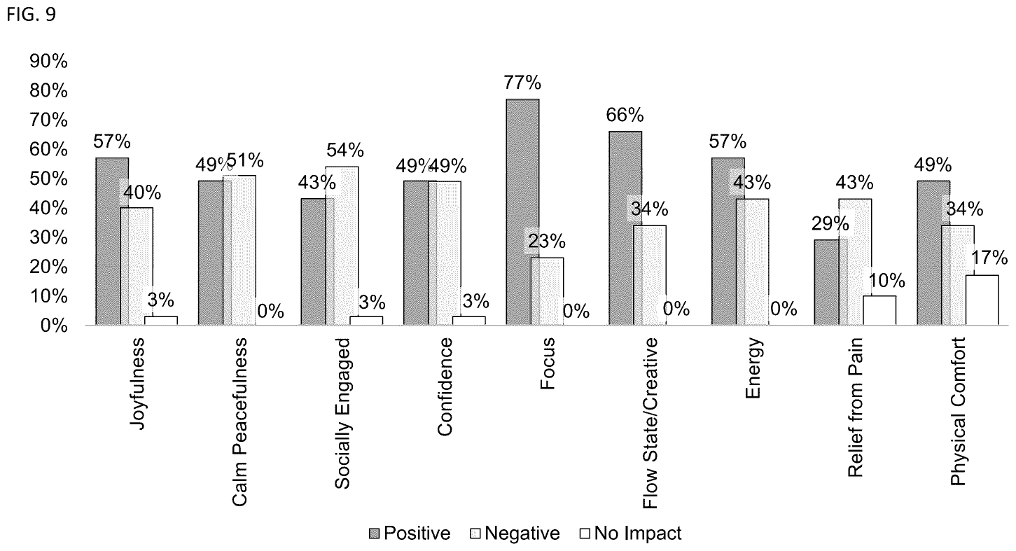

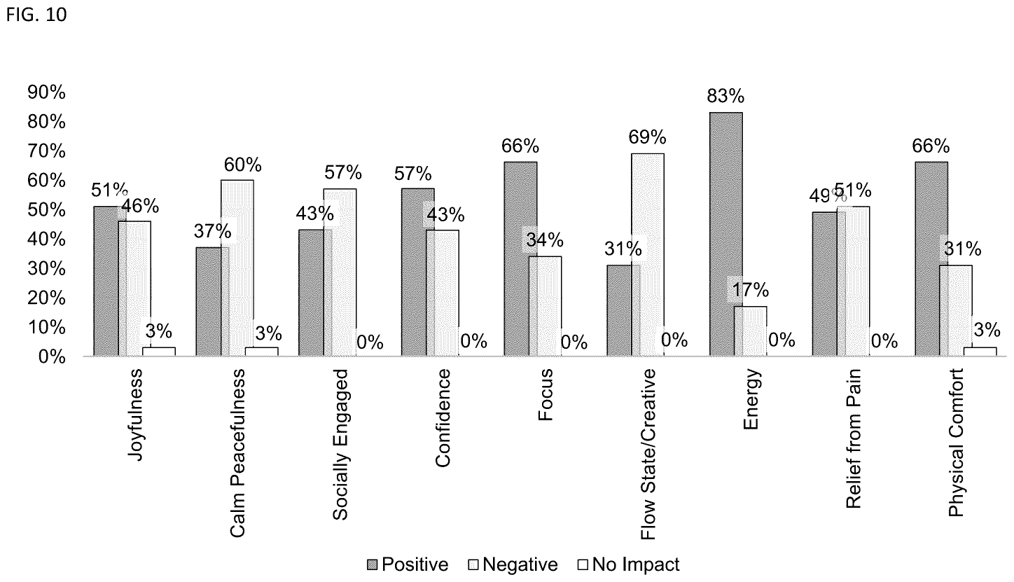

The compositions of the present disclosure also positively affected mental focus when combined with a cannabis product. 77% of the subjects reported improvements to focus; 66% increased creativity; and 57% reported increased energy (FIG. 9). Additionally, the compositions of the present disclosure positively affected mental and physiological energy when combined with a cannabis product. Approximately 83% of the subjects who used the energy boost formulation: stamina and motivation reported an increase in energy; 66% experienced increase in focus; and 66% of the subjects had increase in physical comfort (FIG. 10)

1. A nutritional composition comprising one or more adaptogens, medicinal botanicals, nutraceuticals, or a combination thereof, wherein the nutritional composition is selected from the group consisting of:
(a) a composition comprising about 40 mg to about 300 mg of Panax ginseng, about 100 mg to about 400 mg of acetyl-L-carnitine, about 100 mg to about 400 mg of citicoline, and about 100 mg to about 300 mg of L-theanine;
(b) a composition comprising about 200 mg to about 700 mg of Withania somnifera, about 100 mg to about 300 mg of Corydalis yanhusuo, about 30 mg to about 140 mg of Silybum mariamim, and about 20 mg to about 200 mg of Griffonia simplicifolia;
(c) a composition comprising about 100 mg to about 400 mg of Rhodiola rosea, about 90 mg to about 270 mg of Paullinia cupana, and about 5 mg to about 200 mg of Sceletium tortuosum; and
(d) a composition comprising about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia.

2-31. (canceled)

32. The nutritional composition of claim 1, wherein the composition comprises about 40 mg to about 300 mg of Panax ginseng, about 100 mg to about 400 mg of acetyl-L-carnitine, about 100 mg to about 400 mg of citicoline, and about 100 mg to about 300 mg of L-theanine.

33. A method of increasing mental focus in a subject, comprising administering to the subject the composition of claim 32.

34. The method of claim 33, further comprising administering to the subject a composition comprising one or more cannabinoids.

35. The nutritional composition of claim 1, wherein the composition comprises about 200 mg to about 700 mg of Withania somnifera, about 100 mg to about 300 mg of Corydalis yanhusuo, about 30 mg to about 140 mg of Silybum marianum, and about 20 mg to about 200 mg of Griffonia simplicifolia.

36. A method of reducing environmental, physical, and mental stress in a subject, comprising administering to the subject the composition of claim 35.

37. The method of claim 36, further comprising administering to the subject a composition comprising one or more cannabinoids.

38. The nutritional composition of claim 1, wherein the composition comprises about 100 mg to about 400 mg of Rhodiola rosea, about 90 mg to about 270 mg of Paullinia cupana, and about 5 mg to about 200 mg of Sceletium tortuosum.

39. A method of increasing stamina and motivation in a subject, comprising administering to the subject the composition of claim 38.

40. The method of claim 39, further comprising administering to the subject a composition comprising one or more cannabinoids.

41. The nutritional composition of claim 1, wherein the composition comprises about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia.

42. A method of increasing sociability in a subject, comprising administering to the subject the composition of claim 41.

43. The method of claim 42, further comprising administering to the subject a composition comprising one or more cannabinoids.

44. A method of enhancing the effects of one or more cannabinoids in a subject, comprising administering to the subject a nutritional composition comprising one or more adaptogens, medicinal botanicals, nutraceuticals, or a combination thereof, wherein the nutritional composition is selected from the group consisting of:
(a) a composition comprising about 40 mg to about 300 mg of Panax ginseng, about 100 mg to about 400 mg of acetyl-L-carnitine, about 100 mg to about 400 mg of citicoline, and about 100 mg to about 300 mg of L-theanine;
(b) a composition comprising about 200 mg to about 700 mg of Withania somnifera, about 100 mg to about 300 mg of Corydalis yanhusuo, about 30 mg to about 140 mg of Silybum marianum, and about 20 mg to about 200 mg of Griffonia simplicifolia;
(c) a composition comprising about 100 mg to about 400 mg of Rhodiola rosea, about 90 mg to about 270 mg of Paullinia cupana, and about 5 mg to about 200 mg of Sceletium tortuosum; and
(d) a composition comprising about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia.

45. The method of claim 44, wherein the nutritional composition comprises about 40 mg to about 300 mg of Panax ginseng, about 100 mg to about 400 mg of acetyl-L-carnitine, about 100 mg to about 400 mg of citicoline, and about 100 mg to about 300 mg of L-theanine.

46. The method of claim 44, wherein the nutritional composition comprises about 200 mg to about 700 mg of Withania somnifera, about 100 mg to about 300 mg of Corydalis yanhusuo, about 30 mg to about 140 mg of Silybum mariamim, and about 20 mg to about 200 mg of Griffonia simplicifolia.

47. The method of claim 44, wherein the nutritional composition comprises about 100 mg to about 400 mg of Rhodiola rosea, about 90 mg to about 270 mg of Paullinia cupana, and about 5 mg to about 200 mg of Sceletium tortuosum.

48. The method of claim 44, wherein the nutritional composition comprises about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia.

49. The method of claim 44, wherein the one or more cannabinoids are tetrahydrocannabinol (THC), cannabidiol (CBD), cannabigerol (CBG), cannabichromene (CBC), cannabichromenic acid (CBCA), cannabichromevarinic acid (CBCVA), cannabinol (CBN), cannabichromanon (CBCN), delta-9-tetrahydrocannabinolic acid A (THCA), delta-9-tetrahydrocannabinolic acid B (THCB), delta-9-tetrahydrocannabivarin (THCV), cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromevarin, cannabidolic acid, cannabidiol monomethylether, cannabidiol-C4, cannabidivarinic acid, cannabidiorcol, delta-9-tetrahydrocannabinolic acid-C4, delta-9-tetrahydrocannabivarinic acid, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, trihydroxy-delta-9-tetrahydrocannabinol, or any combination thereof.

50. A method of alleviating hypothalamic-pituitary-adrenal (HPA) axis dysfunction in a subject, comprising administering to the subject a nutritional composition comprising one or more adaptogens, medicinal botanicals, nutraceuticals, or a combination thereof, wherein the nutritional composition is selected from the group consisting of:
(a) a composition comprising about 40 mg to about 300 mg of Panax ginseng, about 100 mg to about 400 mg of acetyl-L-carnitine, about 100 mg to about 400 mg of citicoline, and about 100 mg to about 300 mg of L-theanine;
(b) a composition comprising about 200 mg to about 700 mg of Withania somnifera, about 100 mg to about 300 mg of Corydalis yanhusuo, about 30 mg to about 140 mg of Silybum marianum, and about 20 mg to about 200 mg of Griffonia simplicifolia;
(c) a composition comprising about 100 mg to about 400 mg of Rhodiola rosea, about 90 mg to about 270 mg of Paullinia cupana, and about 5 mg to about 200 mg of Sceletium tortuosum; and
(d) a composition comprising about 200 mg to about 700 mg of Withania somnifera, about 250 mg to about 400 mg of Schisandra chinensis, about 100 mg to about 250 mg of Ilex paraguariensis, and about 20 mg to about 200 mg of Griffonia simplicifolia.
